SEQUENCE LISTING (9 sequences)

SEQ ID NO: 1 - DNA, 4216 bp, Candida albicans
ttttctattctaaccaccctctgcaaattttgaattcaaattttaaagctgtgcatatactacaaaccactttttagacctttcacatttaaaaacactttcgggtagggtttttgtgtcttgtggttaagtttttaaacagaaataaaaaatataaccaaattcaacaattatgcagcctaaggattttatcacttggttttttatttttaaactgaaactactttttttagaaaatgggaattaatgggcaatagaagtgatatgcatatggctaaaagtgttttgtgacatgattagtatacttgtgttttgtttgtttggttttgttcccaatttctcattgccttagtcaggtagaaatttacaaaggcgacaaaaaccaagaagcagaataagttaaatgaagtaaagaaaaaaaaataaagagagagtaaaaaaaaaaatgcacctcaaatacaaacaacatcaaatacaaaatatgaattagacagggaccttgacttcccttaattttaattcttgaaaataatatataagtacatggatatatccccgtgatttcttccaatttgatttttatttattttcaatttaatcaattcacatcaattagtttatatttactattaatacctcacaccaactcacacgattcccactccaatctagctgccgcttgttttaatttgtatccgctagttttttattatttttgtttttttcattgatactgtcaatactattattttgaatagactgtggtatcaatttctatttctaactaactatcactgttaattacaccctttatcgaacttatcacgtggttgctgtcaattcctatttttgtttgcaatctcacatttgtttgtttgttgttgttttttttttttcccttttataaattatccgttaagtttaccgcttttttttttgactattttaaattatgatgtcgcaagctactcctagtgctactcccgtgagaaatgccgatggacaaaagaaaggcaagaagcttccattaattgttgatgttgcaattggtaataatggtaagcaactttaccaggtacacgaatcttccaagatggtaagaaaggaaatgccaagatctaccaattttgctttgaatgatgacatttctgatgaagggttttttataagccaaataaatgaacaaagaactcccattagaaagactttgggtactttgtctccatctagtctcaaccaaaagagaatccgccatgatgtgtacgaccaaggagatgacaatagcaatgacgataattgccaaaatgatgtttatgagcaacaacaacaacaaccacagcaacaacaacaacaacaacaacaacttcatcaacctaatatgtatgagaacggcaacgttggggttatttccaatgatccagtgtcttctgttggccactatgacaacaacccagtttcacatgtacctactggtgcttattcacgtgtccaagacactgacatctggtcagatgatgttgaagaagcttttgaagaagtgttgagacttattccaaagagtggtttgaacaaaataaagattgccggtagatcttgtggtagaaatgaattaatatcagattacatttttgccaaaaccgggaaattcagaacaagaaagcaagtgtcctctcacattcaagtgattaagaatttaggacaaaaactcgatataatccaattgatcaacgatggtcctattttcaacagtcacgaggaacaattggaatcaaccaagaaatttgaagatgttttttcgaaaattaatttgaacaagtcacttgggtttagtgactcgatgaaaagaaaaagtgattcaatgcctatgcatctcccagccacaaagagaataagaagaaaacactcgggcaatcctttgaataaaataaaatttagcaatttcttcatgtctgttaatgaccaatacggtatgaatccaattgttttgacgatacagcaaaatggcaatgacgttaaaagtttgaaattgaaagacaatgctaatatttccagtaggttccctggtttaagtgattttaaatcgtgtcctcatattccaatcattcacaacatggtgaaaatattgctcccgcagttgccagaatcatatagtatcgacgacgggtttagttccagttatgctttgaagtacgaagaaccagaaaacgcatcacccacacataccagtattatatcatcttctagaacttacagcttgttcacttgtgtatattcctatggtaaagagattgtgaaatttgatgaagacggtattcagttgaatcaagacagagaatttataccgggattttggaagttcttcttcagtacatttggggatcaaagtgaaggtggtcttagtgctgctttcaaaggtgtcaccattaaacaaatactttacgagtcaagccccgatagtgttaaaaaggaacaagatgctagtaaagttaataaactgaaagtcaaattggttttactttgggaatttgctaaagtgagtgaatgcaaagatgctttgactactactacaaagttggtgttaccaccacgagcactggcttcaagctcaaagaccactgaagaagtgtttgaatactcagaaccagcattaaactcaattggtggtaccccaacggacactactagtcctaatatggatttgaataatcagaacttgagtgcagcagcaacttcaattcctggtatacgagacaccattcattcagcatcaatgccagatatcaatgaattgccatcttcagctaaaccacaagtaaggttacaaaagactttccaatcgatgcaacacttgcaaccacaccaaatgtggcaacaacaacaacaacaacagccactgcaaggtgcatataccagctcagtagcttcacaactgctcaatacttcattatcatcaccttatgctcaatatggcatgccactaccacaacaaacaataggcacttttgttccacctacttcacaaacatttggtgtttcctacacgcataatagccaacacccttcagctaatatggatcttatgatgctttcttctatgaataccggatatggaaatatcacgaataatcaagattatcaatttggtaatataggatacacagaaggatttactagtgagttttagtgaacaaaatgagaaaagagaagaaaatattatcacttactcactgttggatacattagtttagcggctatacaatctttaattgcatatctttttcttttttttggttggaaaaataaatatagaacagaaataaaattttattggttcgattgtaaatgatgatgaagctttggttcatcttgttcataattgatttggtgtttttttattattattattgtattttcttttttagttcttcgtgatttatatttaattgtgtattttgttttgttttgttttgtttctatataatttattaaatgttgtatgatatatttcttttttgttgtattgttgtaattgcttgtttcataaacaagagatattgtttaatagctcaacaaatctatatgtttcagattattatttgttatcatcgaagtataaagaggtatcttcaatattgaagatgctggcgatgaataaataggggaaaggggggaggtgggaggttattgagcattttgtagttgaagataacccataatgatgatcattgtaaatcatctatgatttaattatctgagtacagaaaagagtgtaatcttgcaaatcgtttctcattatttttttttgggtgatttttatttttgtgattctttttttgtttcgattccattcgtttcgattattattgttatcatcataatggattacataattgtatgaggcttgtattagagtttcaacttagaatgtttgaagtttttttgggtttcttttgtttccacttctttgcattacagttgtttgtactatgtcaaacaggcaagcttgttcaatgcatcaatgcctataagggtccaattttaaaaaatggctattgtaattgtgttttaacagttgaatgtctttttgaatcagtaaacccacaccattgcttgcttgcaaaacaactcctcggtttttacaaacttaaaacttaatgtacagtaaatgaaggggagggtaaagctgaacagcaaaggggaagaggggcaaggcggg

SEQ ID NO: 2 - DNA, 2229 bp, Candida albicans
atgatgtcgcaagctactcctagtgctactcccgtgagaaatgccgatggacaaaagaaaggcaagaagcttccattaattgttgatgttgcaattggtaataatggtaagcaactttaccaggtacacgaatcttccaagatggtaagaaaggaaatgccaagatctaccaattttgctttgaatgatgacatttctgatgaagggttttttataagccaaataaatgaacaaagaactcccattagaaagactttgggtactttgtctccatctagtctcaaccaaaagagaatccgccatgatgtgtacgaccaaggagatgacaatagcaatgacgataattgccaaaatgatgtttatgagcaacaacaacaacaaccacagcaacaacaacaacaacaacaacaacttcatcaacctaatatgtatgagaacggcaacgttggggttatttccaatgatccagtgtcttctgttggccactatgacaacaacccagtttcacatgtacctactggtgcttattcacgtgtccaagacactgacatctggtcagatgatgttgaagaagcttttgaagaagtgttgagacttattccaaagagtggtttgaacaaaataaagattgccggtagatcttgtggtagaaatgaattaatatcagattacatttttgccaaaaccgggaaattcagaacaagaaagcaagtgtcctctcacattcaagtgattaagaatttaggacaaaaactcgatataatccaattgatcaacgatggtcctattttcaacagtcacgaggaacaattggaatcaaccaagaaatttgaagatgttttttcgaaaattaatttgaacaagtcacttgggtttagtgactcgatgaaaagaaaaagtgattcaatgcctatgcatctcccagccacaaagagaataagaagaaaacactcgggcaatcctttgaataaaataaaatttagcaatttcttcatgtctgttaatgaccaatacggtatgaatccaattgttttgacgatacagcaaaatggcaatgacgttaaaagtttgaaattgaaagacaatgctaatatttccagtaggttccctggtttaagtgattttaaatcgtgtcctcatattccaatcattcacaacatggtgaaaatattgctcccgcagttgccagaatcatatagtatcgacgacgggtttagttccagttatgctttgaagtacgaagaaccagaaaacgcatcacccacacataccagtattatatcatcttctagaacttacagcttgttcacttgtgtatattcctatggtaaagagattgtgaaatttgatgaagacggtattcagttgaatcaagacagagaatttataccgggattttggaagttcttcttcagtacatttggggatcaaagtgaaggtggtcttagtgctgctttcaaaggtgtcaccattaaacaaatactttacgagtcaagccccgatagtgttaaaaaggaacaagatgctagtaaagttaataaactgaaagtcaaattggttttactttgggaatttgctaaagtgagtgaatgcaaagatgctttgactactactacaaagttggtgttaccaccacgagcactggcttcaagctcaaagaccactgaagaagtgtttgaatactcagaaccagcattaaactcaattggtggtaccccaacggacactactagtcctaatatggatttgaataatcagaacttgagtgcagcagcaacttcaattcctggtatacgagacaccattcattcagcatcaatgccagatatcaatgaattgccatcttcagctaaaccacaagtaaggttacaaaagactttccaatcgatgcaacacttgcaaccacaccaaatgtggcaacaacaacaacaacaacagccactgcaaggtgcatataccagctcagtagcttcacaactgctcaatacttcattatcatcaccttatgctcaatatggcatgccactaccacaacaaacaataggcacttttgttccacctacttcacaaacatttggtgtttcctacacgcataatagccaacacccttcagctaatatggatcttatgatgctttcttctatgaataccggatatggaaatatcacgaataatcaagattatcaatttggtaatataggatacacagaaggatttactagtgagttt

SEQ ID NO: 3 - DNA, 952 bp, Candida albicans
ttttctattctaaccaccctctgcaaattttgaattcaaattttaaagctgtgcatatactacaaaccactttttagacctttcacatttaaaaacactttcgggtagggtttttgtgtcttgtggttaagtttttaaacagaaataaaaaatataaccaaattcaacaattatgcagcctaaggattttatcacttggttttttatttttaaactgaaactactttttttagaaaatgggaattaatgggcaatagaagtgatatgcatatggctaaaagtgttttgtgacatgattagtatacttgtgttttgtttgtttggttttgttcccaatttctcattgccttagtcaggtagaaatttacaaaggcgacaaaaaccaagaagcagaataagttaaatgaagtaaagaaaaaaaaataaagagagagtaaaaaaaaaaatgcacctcaaatacaaacaacatcaaatacaaaatatgaattagacagggaccttgacttcccttaattttaattcttgaaaataatatataagtacatggatatatccccgtgatttcttccaatttgatttttatttattttcaatttaatcaattcacatcaattagtttatatttactattaatacctcacaccaactcacacgattcccactccaatctagctgccgcttgttttaatttgtatccgctagttttttattatttttgtttttttcattgatactgtcaatactattattttgaatagactgtggtatcaatttctatttctaactaactatcactgttaattacaccctttatcgaacttatcacgtggttgctgtcaattcctatttttgtttgcaatctcacatttgtttgtttgttgttgttttttttttttcccttttataaattatccgttaagtttaccgcttttttttttgactattttaaatt

SEQ ID NO: 4 - protein, 743 aa, Candida albicans
MMSQATPSATPVRNADGQKKGKKLPLIVDVAIGNNGKQLYQVHESSKMVRKEMPRSTNFALNDDISDEGFFISQINEQRTPIRKTLGTLSPSSLNQKRIRHDVYDQGDDNSNDDNCQNDVYEQQQQQPQQQQQQQQQLHQPNMYENGNVGVISNDPVSSVGHYDNNPVSHVPTGAYSRVQDTDIWSDDVEEAFEEVLRLIPKSGLNKIKIAGRSCGRNELISDYIFAKTGKFRTRKQVSSHIQVIKNLGQKLDIIQLINDGPIFNSHEEQLESTKKFEDVFSKINLNKSLGFSDSMKRKSDSMPMHLPATKRIRRKHSGNPLNKIKFSNFFMSVNDQYGMNPIVLTIQQNGNDVKSLKLKDNANISSRFPGLSDFKSCPHIPIIHNMVKILLPQLPESYSIDDGFSSSYALKYEEPENASPTHTSIISSSRTYSLFTCVYSYGKEIVKFDEDGIQLNQDREFIPGFWKFFFSTFGDQSEGGLSAAFKGVTIKQILYESSPDSVKKEQDASKVNKLKVKLVLLWEFAKVSECKDALTTTTKLVLPPRALASSSKTTEEVFEYSEPALNSIGGTPTDTTSPNMDLNNQNLSAAATSIPGIRDTIHSASMPDINELPSSAKPQVRLQKTFQSMQHLQPHQMWQQQQQQQPLQGAYTSSVASQLLNTSLSSPYAQYGMPLPQQTIGTFVPPTSQTFGVSYTHNSQHPSANMDLMMLSSMNTGYGNITNNQDYQFGNIGYTEGFTSEF

SEQ ID NO: 5 - DNA, 24 bp, Candida albicans
ttttctattctaaccaccctctgc

SEQ ID NO: 6 - DNA, 17 bp, Candida albicans
cccgccttgcccctctt

SEQ ID NO: 7 - DNA, 1032 bp, Candida albicans
tgaacaaaatgagaaaagagaagaaaatattatcacttactcactgttggatacattagtttagcggctatacaatctttaattgcatatctttttcttttttttggttggaaaaataaatatagaacagaaataaaattttattggttcgattgtaaatgatgatgaagctttggttcatcttgttcataattgatttggtgtttttttattattattattgtattttcttttttagttcttcgtgatttatatttaattgtgtattttgttttgttttgttttgtttctatataatttattaaatgttgtatgatatatttcttttttgttgtattgttgtaattgcttgtttcataaacaagagatattgtttaatagctcaacaaatctatatgtttcagattattatttgttatcatcgaagtataaagaggtatcttcaatattgaagatgctggcgatgaataaataggggaaaggggggaggtgggaggttattgagcattttgtagttgaagataacccataatgatgatcattgtaaatcatctatgatttaattatctgagtacagaaaagagtgtaatcttgcaaatcgtttctcattatttttttttgggtgatttttatttttgtgattctttttttgtttcgattccattcgtttcgattattattgttatcatcataatggattacataattgtatgaggcttgtattagagtttcaacttagaatgtttgaagtttttttgggtttcttttgtttccacttctttgcattacagttgtttgtactatgtcaaacaggcaagcttgttcaatgcatcaatgcctataagggtccaattttaaaaaatggctattgtaattgtgttttaacagttgaatgtctttttgaatcagtaaacccacaccattgcttgcttgcaaaacaactcctcggtttttacaaacttaaaacttaatgtacagtaaatgaaggggagggtaaagctgaacagcaaaggggaagaggggcaaggcggg

SEQ ID NO: 8 - DNA, 31 bp, Candida albicans
tttaggatccaatgatgtcgcaagctactcc

SEQ ID NO: 9 - DNA, 37 bp, Candida albicans
tttaggatccactaaaactcactagtaaatccttctg

(19) United States
(12) Patent Application Publication
(10) Pub. No.: US 2004/0044193 A1
(43) Pub. Date: Mar. 4, 2004

(54) C. albicans tec1 gene (catec1) and the coded tecip protein

(76) Inventors: Klaus Schroppel, Kleinseldelbach (DE); Brad N. Taylor, Erlangen (DE); Anja Schweizer, Erlangen (DE); Steffen Rupp, Stuttgart (DE)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/380,334

(22) PCT Filed: Sep. 13, 2001
(86) PCT No.: PCT/EP01/10596

(30) Foreign Application Priority Data
Sep. 13, 2000 (DE) 10045123.3

(51) Int. Cl. C07H 21/02; C07H 21/04

(52) U.S. Cl. 536/23.1

(57) ABSTRACT

The present invention relates to nucleotide sequences which code for proteins modulating the virulence of pathogenic fungal strains; vectors which contain these sequences; host cells which have these nucleotide sequences or vectors; proteins modulating the virulence of pathogenic fungal strains, in particular the transcription factor CaTEC1 of Candida albicans; inhibitors thereof, particularly antibodies which are directed against these virulence-modulating proteins; methods for the production of the proteins and inhibitors; methods for the diagnosis and therapy of diseases which are connected with a Candida infection; and also diagnostic and pharmaceutical compositions which contain the nucleotide sequences, proteins, host cells and/or antibodies.

### FIELD OF THE INVENTION

The present invention relates to nucleic acid molecules which code for proteins modulating the virulence of pathogenic fungal strains; vectors which contain these molecules; host cells which have these nucleic acid molecules or vectors; proteins modulating the virulence of pathogenic fungal strains; inhibitors of the proteins and of the nucleic acid molecules coding for the proteins, particularly antibodies which are directed against these virulence-modulating proteins; methods of preparing the proteins and antibodies; and also diagnostic and pharmaceutical compositions which contain these nucleotide sequences, proteins, host cells, and/or antibodies.

### BACKGROUND OF THE INVENTION

Yeasts are fungi which proliferate vegetatively by budding or division. Besides the yeasts of commercial use of the family Saccharomycetaceae, the budding fungi or yeasts also include species pathogenic to humans, for example, Candida albicans. Candida albicans is a thin-walled, gram-positive, capsule-less yeast of oval to round shape, which is a facultative pathogen for humans, porpoises, mice, rats and birds. Candida albicans is the most frequent source of superficial Candida mycoses in humans. Furthermore, Candida albicans frequently causes opportunistic infections, i.e., infections by normally relatively unproblematic germs, in immunosuppressed patients. In such immunosuppressed patients, such infections can take a difficult course and can decisively shorten the life of, for example, HIV-infected or cancer patients who are treated with chemotherapy or radiotherapy. Systemic infections brought about by Candida albicans are at present mainly treated with the aid of azoles or polyenes. While polyenes have strong side effects, increasing resistances develop against azoles (DiDomenico, Curr. Opin. Microbiol., 2 (1999), 509-515; Georgopapadakou, Curr. Opin. Microbiol., 1 (1998), 547-557).

It is therefore imperative to develop further improved antimycotics for the treatment of infections which are brought about by representatives of the Candida family.

It is known that the morphological development of fungi and higher eukaryotes is regulated by transcription factors which control the expression of genes which are specific for given stages of development. The TEA/ATTS transcription factor family belongs to these transcription factors and has been found in mammals, birds, nematodes, insects, and fungi. All the representatives of this transcription factor family have a conserved TEA domain. This TEA domain (Bürglin, Cell, 66 (1991), 11-12) represents a DNA binding region which consists of 66-76 conserved amino acids in the N-terminal region of the protein. The TEA consensus sequence (TCS) in the target promoters of fungal TEA/ATTS transcription factors is defined by the sequence 5′-CATTCY-3′ (Adrianopoulos and Timberlake, Mol. Cell. Biol., 14 (1994), 2503-2515). It is striking that for all representatives of this transcription factor family they play a decisive role in development and organogenesis. For example, the mammalian enhancer factor TEF-1 takes part in myogenesis and cardiogenesis (Jacquemin et al., J. Biol. Chem., 271 (1996), 21775-21785). The Drosophila melanogaster protein “scalloped” takes part in the regulation of cell-specific gene expression during the development of the wings and nervous system (Campbell et al., Genes Dev., 6 (1992), 367-379). The regulating protein “abacus” (AbaAp) of Aspergillus nidulans takes part in the regulation of conidium formation, that is, its expression leads to the ending of the vegetative growth phase. The in trans effective factor Tec1p of Saccharomyces cerevisiae takes part in the activation of the Ty1 retrotransposon (Laloux et al., Mol. Cell. Biol., 10 (1990), 3541-3550). Likewise, this factor takes part in the regulation of filament formation and the invasive growth of haploid and diploid strains of yeast (Baur et al., Mol. Cell. Biol., 17 (1997), 4330-4337).

Because of the importance of the TEA/ATTS transcription factor for morphological development, that is, the spatial and temporal development of organisms or respectively of their organs or tissues, and their further propagation in the most varied kinds, it appears possible to identify such transcription factors, or the genes underlying them, in pathogenic microorganisms as well, in human-pathogenic fungi in particular, and to use them as targets for the identification of substances which act specifically on these transcription factors or the nucleotide sequences coding for them.

The technical problem upon which the present invention is based thus consists of providing means for the development of diagnostically and therapeutically active substances, particularly of substances which are specifically directed against the TEA/ATTS transcription factors of the yeast Candida albicans, and also further means and methods, based on these means, which can be used for the diagnosis and therapy of infections or disease states brought about by Candida albicans.

The invention solves its basic technical problem by the provision of nucleic acid molecules which are suitable for the cloning of a gene for a transcription factor, in particular for a protein from Candida, particularly Candida albicans, modifying the virulence of human-pathological fungi, and/or the recombinant production of such a protein, these nucleic acid molecules being chosen from the group consisting of:

(a) a nucleic acid molecule, defined in SEQ ID No. 1, 2, 3 or 7, and/or a complementary strand or portion thereof;

(b) a nucleic acid molecule, coding for an amino acid sequence defined in SEQ ID No. 4 or a complementary strand or portion thereof;

(c) a nucleic acid molecule, obtainable from a gene bank of Candida albicans using the primer represented in SEQ ID No. 5 and SEQ ID No. 6 by means of the PCR method; and

(d) a nucleic acid molecule which, based on its homology, hybridizes with one of the nucleic acid molecules named in (a)-(c), the homology on the nucleotide level (sequence identity) specifically amounting to at least 65%, preferably at least 70%, and most preferably at least 90%, 95%, 96%, 97%, 98%, 99%.

The gene represented by the nucleic acid molecule according to the invention of the SEQ ID No. 1 is also denoted as the CaTEC1 gene in what follows. The CaTEC1 gene according to the invention is expressed in vivo both in the yeast form and in mycelia of C. albicans.

The nucleic acid molecules according to the invention are in particular characterized in that after introduction into Candida albicans-CaTEC1 mutants, for example by means of a vector, in which they are integrated in sense orientation under the control of suitable regulating elements, they can revert the phenotype of such yeast mutants.

The phenotype of CaTEC1 mutants, for example, the homozygotic ura3/ura3 CaTEC1/CaTEC1 null mutant CaAS15 (null mutant phenotype), differs from the phenotype of wild-type cells, particularly in so far as the filamentary growth in vitro is considered, the formation of germ tubes is suppressed, the expression of SAP4-6 genes cannot be induced, and the mutant shows a reduced virulence in a mouse Candida mycosis model. The said phenotypes thus characterize the biological activity of the CaTEC1 coded protein (CaTec1p).

The proteins coded for by the nucleic acid molecules according to the invention represent transcription factors which control and regulate the expression of development-specific genes and thus the morphological development of Candida albicans cells.

The nucleotide and amino acid sequences particularly occurring in pathogenic wild-type forms of Candida albicans thus represent excellent auxiliaries for reducing and/or eliminating the virulence of pathogenic Candida albicans strains. The nucleic acid and amino acid molecules according to the invention are thus found to be particularly valuable for the development of medicaments for combating Candida mycoses. The nucleic acid molecules according to the invention and the proteins according to the invention coded for by them can for example be used as targets for the identification of substances which act specifically on the nucleotide sequences according to the invention or on the proteins according to the invention. Thus, for example, substance libraries can be analyzed for the reciprocal action of the substances present in these with the proteins according to the invention or their action on the expression of the nucleic acid molecules according to the invention. Likewise, monoclonal or polyclonal antibodies directed against the proteins according to the invention can be developed which on the one hand can be used for the identification of Candida-specific proteins and hence for the identification of Candida infections, and on the other hand can be directly used for combating such infections when functionalized, for example, with the use of cytotoxic groups, such as cytotoxic proteins or cytotoxic peptides, so that in this manner the target organism can be killed and a therapy can be made possible for the disease states brought about by these organisms.

A preferred embodiment of the invention therefore relates to a nucleic acid molecule according to the invention shown in SEQ ID No. 1, this nucleic acid molecule comprising, besides 5′- and 3′-regulating elements, a protein-coding nucleotide sequence. The nucleotide sequences according to the invention code for the amino acid sequence shown in SEQ ID No. 4. The nucleic acid molecules according to the invention are particularly helpful in the development of new therapeutics insofar as they permit the production of the proteins or derivatives for which they code by means of DNA recombination techniques. Furthermore, the nucleic acid molecules according to the invention can be used in antisense constructs or as complementary strands of the coded nucleotide sequences or as portions thereof, in order to inhibit or decrease the endogenous expression in Candida cells of the nucleotide sequence according to the invention. It is possible in this manner to decrease or eliminate the virulence of such Candida cells. Candida cells which for example contain antisense constructs of the nucleotide sequences according to the invention can, for example, be used as living inoculation material in the context of a protective inoculation, for example in the context of an active immunization, in order to counter later infections with pathogenic kinds of Candida, particularly Candida albicans.

It is likewise possible, for example, to introduce the nucleic acid molecules or portions thereof into Candida cells in order to mutate, and by means of homologous recombination particularly to eliminate, endogenous CaTEC1 genes which are present, and thus to produce null mutants. The invention therefore relates to the use of the nucleic acid molecules according to the invention for the production of mutant phenotypes of yeast fungal cells, particularly Candida cells. Likewise the invention relates to these mutant Candida cells themselves and also the mutated catec1 or CaTEC1 genes present in these cells. Such mutated CaTEC1 or CaTEC1 genes can prove to be very helpful in the development of pharmaceutics and diagnostics for combating diseases brought about by Candida.

The invention also relates to plasmids containing the CaTEC1 gene (SEQ ID No. 1) deposited on Sep. 6, 2000 at the DSMZ in Braunschweig, Germany in Escherichia coli under the number DSM 13716.

The invention also relates to CaTEC1 mutants, specifically the Candida albicans mutant CaAS18, deposited on Sep. 6, 2000 at the DSMZ in Braunschweig, Germany under the number DSM 13722.

In a further preferred embodiment, the invention relates to the said nucleic acid molecules, preferably present in completely or partially isolated and purified form, in a particularly preferred embodiment as DNA or RNA sequences.

The invention likewise includes modifications of the nucleic acid molecules according to the invention which lead to the synthesis of proteins with altered activity for the modulation of the virulence properties of pathogenic fungi, preferably of Candida cells. The modifications or mutations of the nucleotide sequences according to the invention can concern both naturally arising modifications or mutations and also those produced by standard microbiological/molecular biological procedures known to those skilled in the art (cf. Sambrook et al., Molecular Cloning: A Laboratory Manual, 2d. Edition (1989), Cold Spring Harbor Laboratory Press, NY, USA). The mutations or modifications covered by the present invention can be insertions, deletions, duplications, inversions, displacements, exchanges or the like, particularly also of unusual nucleotides. Furthermore, the invention also includes mutations or modifications of the nucleotide sequences according to the invention which are brought about by fusion with genes or components of genes from other sources. The invention particularly includes shortened nucleotide sequences of the said kind, insofar as these code for proteins or portions of proteins which can modulate the virulence of Candida cells. The invention also particularly includes mutations or modifications of nucleotide sequences which lead to proteins which have an altered stability, specificity, a modified temperature, pH value, and/or concentration profile, an altered activity and/or an altered effector pattern, and also those whose conformations are altered, or respectively those which have other subunits or other pre- and/or post-translational modifications.

The invention likewise relates to nucleotide sequences which can hybridize with the abovementioned nucleotide sequences according to the invention. In connection with the invention, a “hybridization” means the apposition of two single-stranded nucleic acid molecules under conditions, as described in Sambrook et al. (Molecular Cloning: A Laboratory Manual, 2d. Edition (1989), Cold Spring Harbor Laboratory Press, NY, USA), where stringent conditions are preferably concerned. The concept “nucleic acid molecule which hybridizes with a nucleic acid molecule according to (a)(c)” used in connection with the present invention therefore refers to a nucleic acid molecule which hybridizes with a nucleic acid according to (a)-(c) under preferably stringent conditions. Such a hybridization is characterized in that after washing for an hour with 1×SSC and 0.1% SDS at 55° C., preferably 62° C. and particularly preferably 68° C., in particular for one hour with 0.2×SSC and 0.1% SDS at 55° C., preferably 62° C. and particularly preferably 68° C., a positive hybridization signal is still to be observed. A nucleotide sequence according to the invention is therefore a nucleotide sequence which hybridizes under such washing conditions with the nucleotide sequence given in the sequence protocols.

The molecules hybridizing with the nucleotide sequences according to the invention also include fragments, derivatives, functional equivalents and/or allelic variants of the nucleotide sequences described hereinabove, which code for a protein according to the invention. In connection with the present invention, “fragments” is to be understood as portions of nucleotide sequences which have a sufficient length to code for the abovementioned protein or an equivalent protein, which has activity for the modulation of virulence properties of Candida cells. The concepts “derivative,” “functional equivalent,” or “variant” mean in connection with the present invention that the sequences of these molecules differ from the sequences of the abovementioned nucleotide sequences in at least one position, but have a high degree of homology with these sequences at the nucleic acid level. “Homology” means in particular a sequence identity of at least 40%, in particular at least 60%, preferably of over 80% and particularly preferred over 90%, 95%, 97% or 99% at the nucleic acid level.

The amino acid sequences of the proteins coded for by these nucleic acid molecules are homologous to the amino acid sequence shown in SEQ ID No. 4, the homology being at least 65%, preferably at least 70%, 80%, and more preferably at least 85%, and particularly preferably at least more than 90%, 95%, 97%, and 99%. In connection with the present invention, the expression “at least 80%, and preferably at least 85%, and particularly preferably at least more than 90%, 95%, 97%, and 99% homologous” relates to a sequence agreement at the amino acid sequence level, which can be determined by means of known methods, for example, computer-supported sequence comparisons (Basic Local Alignment Tool, Altschul et al., J. Mol. Biol., 215 (1990), 403-410). The expression “homology,” known to one skilled in the art, denotes a degree of relationship between two or more polypeptide molecules, as determined by the agreement between the sequences, where “agreement” can mean both an identical agreement and also a conservative amino acid exchange. The percentage degree of homology is given by the percentage degree of agreeing regions between two or more sequences, taking account of gaps or other sequence peculiarities.

The differences from the amino acid sequences according to the invention can arise, for example, from mutations, such as for example deletions, substitutions, insertions, displacement, exchanges and/or recombinations or the nucleotide sequences coding for the amino acid sequences. Of course this can also concern naturally occurring sequence variations, for example, sequences from another organism or sequences which were naturally mutated, or mutations which were deliberately introduced into the sequences by means of conventional means known to one skilled in the art, for example, chemical agents and/or physical agents.

### BRIEF SUMMARY OF THE INVENTION

The present invention therefore likewise relates to a polypeptide or protein with a biological activity which in particular, in Candida albicans in vivo, affects filamentous growth and/or the formation of germ tubes and hyphae proper, induces or inhibits the expression of the proteinase isogene SAP4-6, and/or can modulate the virulence of the Candida albicans cells containing it. The present invention relates in particular to the Candida albicans transcription factor Tec1p, which is also termed CaTEC1 protein in what follows. In connection with the present invention, the concepts “protein or polypeptide modulating the virulence of pathogenic fungal strains” or “activity modulating virulence or virulence properties” means that a protein or polypeptide has an activity which can alter, for example decrease, the degree of aggressiveness of an infectious microorganism in a macro-organism. The activity of the amino acid sequences according to the invention can for example be investigated and quantitatively determined by means of the systemic model of mouse Candida mycoses described hereinbelow.

The present invention likewise relates to a protein, preferably isolated and completely purified, which is obtainable by the expression of a nucleic acid molecule according to the invention, or a fragment thereof, in a host cell, and which has the said biological activity.

### BRIEF DESCRIPTION OF THE DRAWINGS

The invention is explained using the following examples and Figures.

FIG. 1 shows the results or a Northern blot analysis of CaTEC1 expression in C. albicans.

For the Northern blot analysis, RNA was extracted from wild-type species SC5314 during the log growth phase in YPD medium at 28° C. (track 1) or after 45, 90, and 300 minutes of growth at 37° C. in RPM1-1640 liquid culture medium enriched with serum (tracks 2-4). The blots were hybridized with CaTEC1 and with ACT1, which served as a control.

FIG. 2 shows that the CaTEC1 gene of C. albicans is a functional homolog of the TEC1 gene of S. cerevisiae.

(A) shows that CaTEC1 can reinstate the pseudohyphal growth in diploid S. cerevisiae cells which have a TEC1 deletion. The pseudohyphal growth is the strains on SLAD medium with uracil after 48 hours of incubation at 30° C. is shown. The strains were L5791 (TEC1/TEC1), pRS425, pRS313), L6146 (TEC1/TEC1, pRS425) and L6146 (TEC1/TEC1, pRS425 CaTEC1).

(B) shows that CaTEC1 can reinstate the invasive growth of haploid and diploid S. cerevisiae cells with TEC1 deletions. The invasive growth of strains which were incubated for 24 hours at 30° C. on YPID medium is shown before washing (A) and after washing (B). The strains in the upper row are the haploid strain (MAT a) 10560-2B (TEC1), pRS425, pRS313), L6149 (TEC1, pRS425) and L6149 (TEC1, pRS425 CaTEC1). The strains in the lower row are the diploid strains (MAT a/α) L5791 (TEC1/TEC1, PRS425, pRS313), L6146 (TEC1/TEC1, pRS425) and L6146 (TEC1/TEC1, PRS425 CaTEC1).

(C) shows that CaTEC1 induces FL011-lacZ expression in haploid and diploid S. cerevisiae with TEC1 deletions. The strains were grown for 48 hours at 30° C. in SC medium. Thereafter, β-galactosidase tests were performed. The strains used were the haploid (MAT a) strain 10560-2B (TEC1, pRS425, pRS313, B3782), L6149 (TEC1, pRS425, B3782) and L6149 (TEC1, pRS425 CaTEC1, B3782), and the diploid (MAT a/α) strains L5791 (TEC1/TEC1, pRS425, pRS313, B3782), L6146 (TEC1/TEC1, pRS425, B3782) and L6146 (TEC1/TEC1, pRS425 CaTEC1, B3782). The units were normalized with respect to the haploid wild type or the diploid wild type, respectively.

FIG. 3 shows how the CaTEC1 gene in C. albicans was inactivated.

(A) The open reading frame for CaTEC1 between the BgIII and EcoRV interfaces was replaced by a hisG-URA3-hisG deletion cassette. P1 and P2 are oligonucleotide binding sites for TECE gene PCR.01 or respectively TEC1 gene PCR.02, which were used for PCR amplification of the CaTEC1 gene.

Probe: position of the NsiI fragment, which was used as probe for hybridization.

(B) shows a Southern blot of stepwise produced isogene mutants with a CaTEC1 fragment as probe (probe, FIG. 3A). The NsiI-digested DNA originated from the following strains: CA1-4 (CaTEC1/CaTEC1)=track 1; CaAS1, CaAS3, CaAS4 (all CaTEC1/CaTEC1: hisG-URA3-hisG)=tracks 2-4; CaAS12, CaAS13, CaAS14 (all CaTEC1::hisG/CaTEC1::hisG-URA3-hisG)=tracks 5-7; CaAS15 (CaTEC1::hisG/CaTEC1::hisG)=track 8.

(C) shows the Northern analysis of CaTEC1 expression of the revertant CaAS20 (CaTEC1/CaTEC1 pVEC-CaTEC1) and the mutant CaAS18 (CaTEC1/CaTEC1 pVEC) during growth in the log phase in YPD medium at 28° C. (tracks 1, 5) and after growth for 45 minutes (tracks 2, 6), 90 minutes (tracks 3, 7) and 300 minutes (tracks 4, 8) in liquid culture medium which had been enriched with serum, at 37° C. ACT1 expression was analyzed as a control and is shown in the two lower images.

FIG. 4 shows the suppression of hyphae formation by a mutation of the CaTEC1 gene. The strain CaAS20 (CaTEC1/CaTEC1 pVEC-CaTEC1) (left-hand image) and the mutant CaAS18 (CaTEC1/CaTEC1 pVEC) (right-hand image) were inoculated over in RPM1-1640 liquid medium which had been enriched with serum at 37° C., and were tested for the growth of hyphae after 90 minutes (a, d), 3 hours (b, e) and 12 hours (c, f). The arrows in (e) show examples of cell wall constrictions between pseudohyphal cells.

FIG. 5 shows the reciprocal action between C. albicans and primary MΦ cells. The revertant CaAS20 (CaTEC1/CaTEC1 pVEC-CaTEC1) (left-hand image) and the mutant CaAS18 (CaTEC1/CaTEC1 pVEC) (right-hand image) were added to primary MΦ cell cultures for 8 hours (a, b) and 24 hours (c, d). CaAS20 goes around the MΦ and forms germ tubes and long filaments, while CaAS18 is concentrated in MΦ (arrow in d).

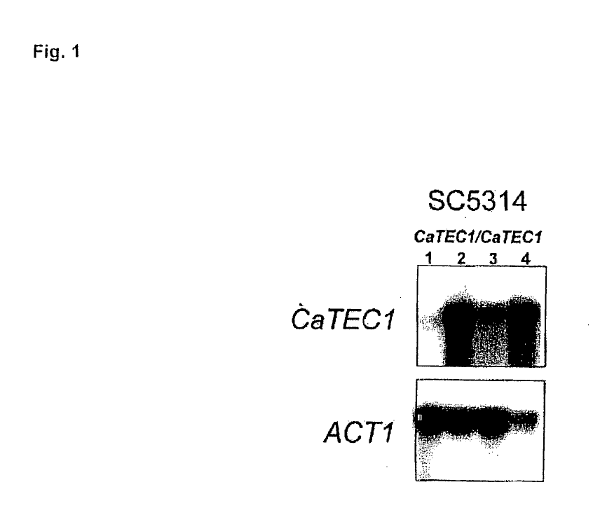

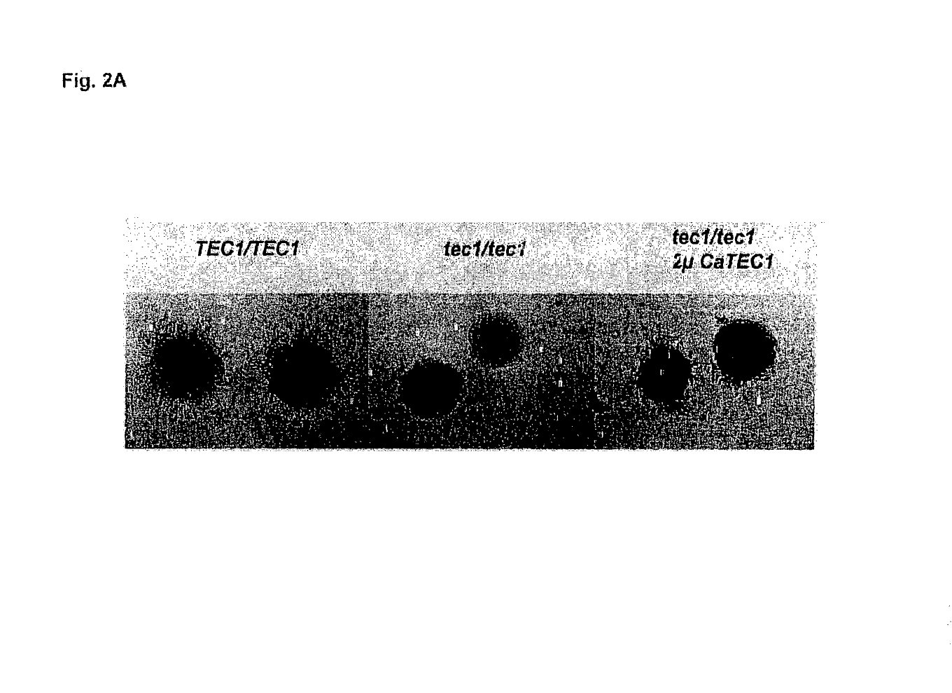

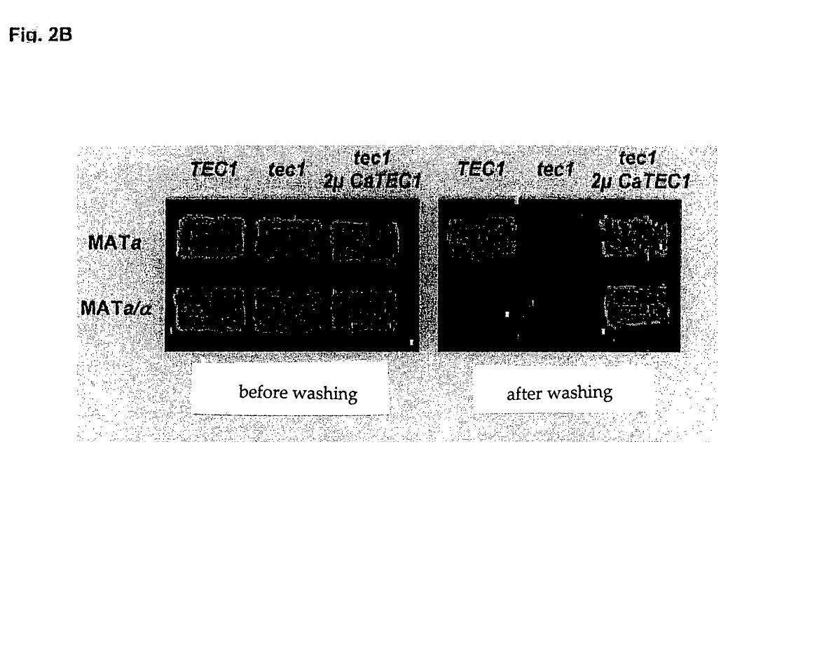

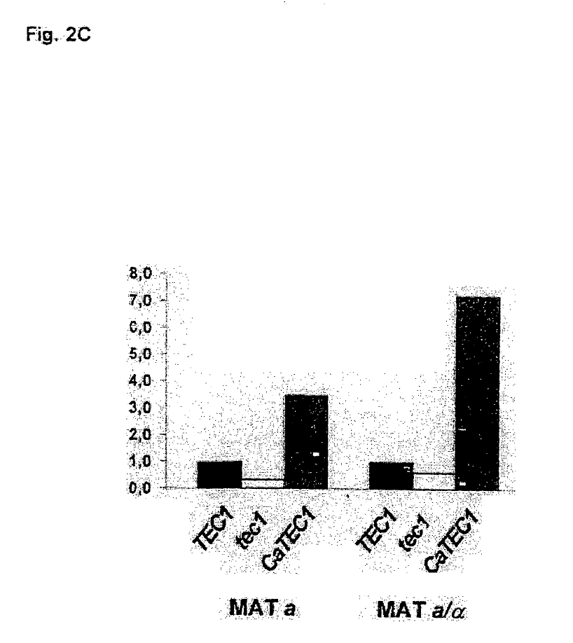

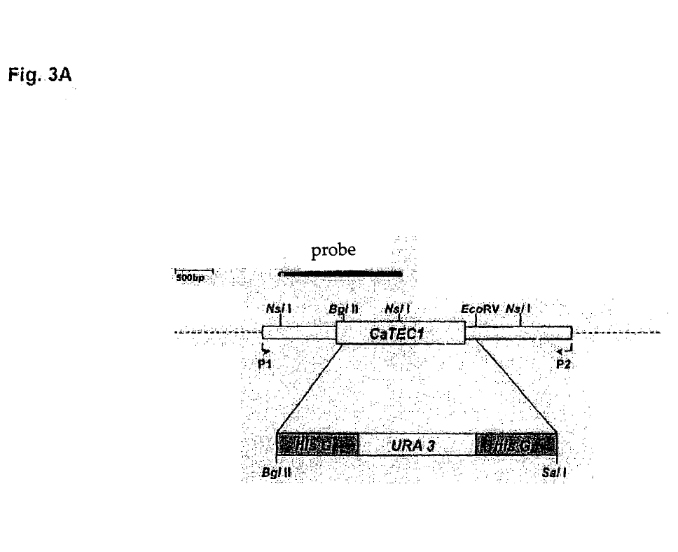

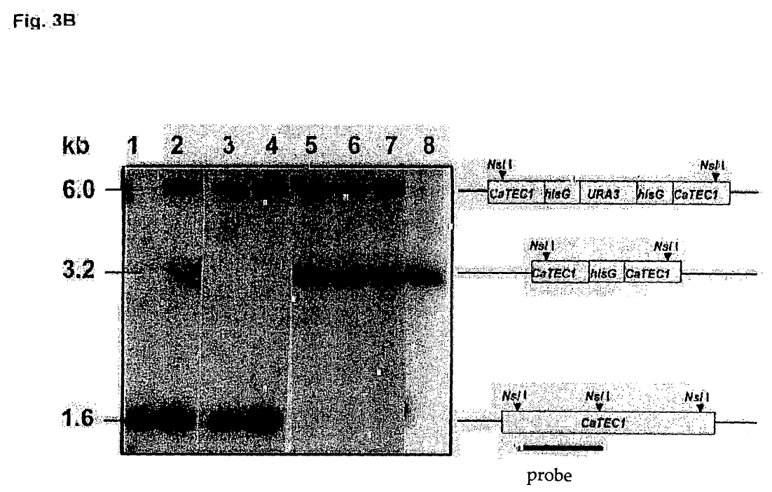

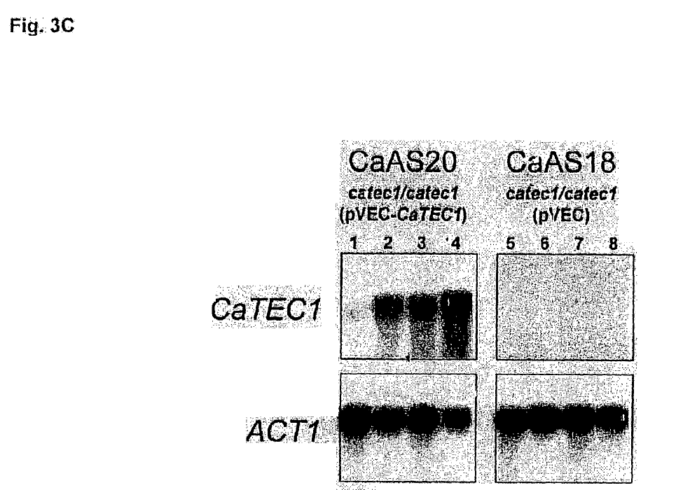

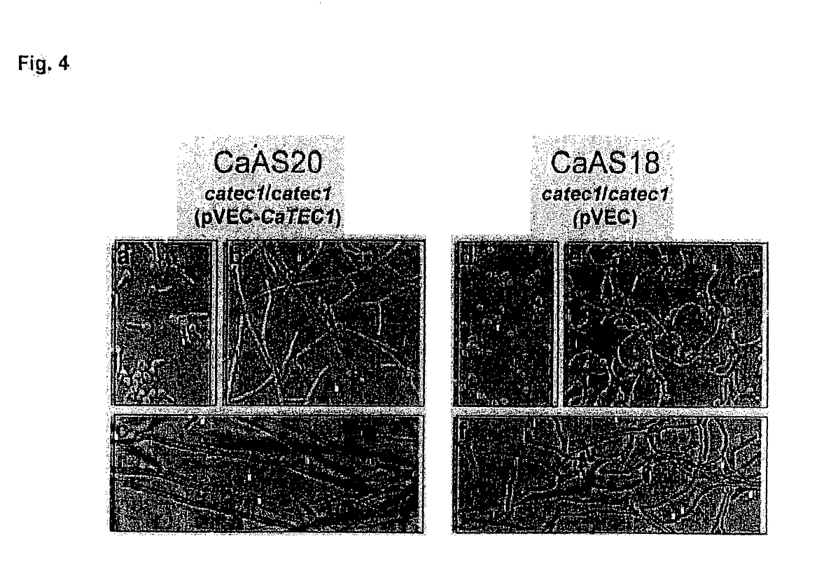

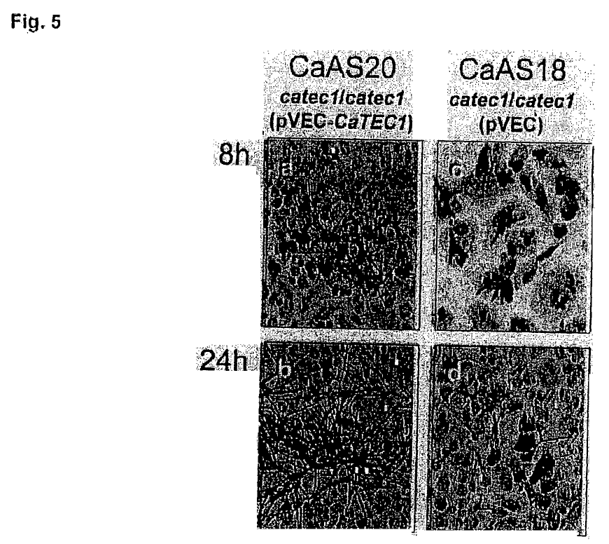

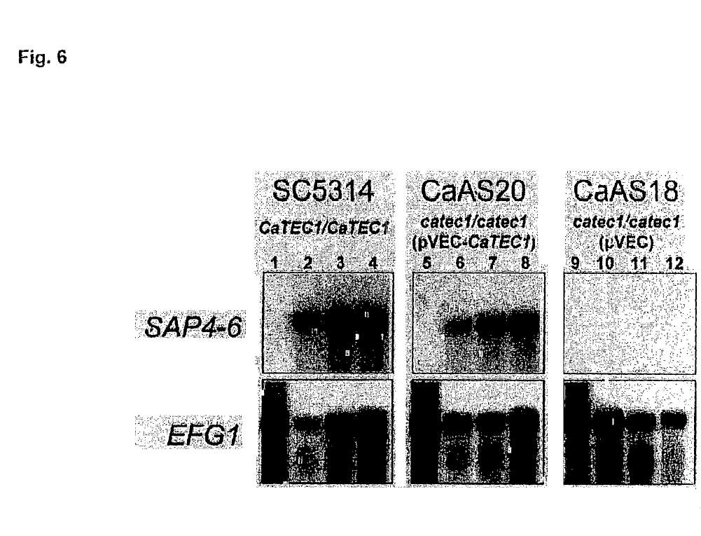

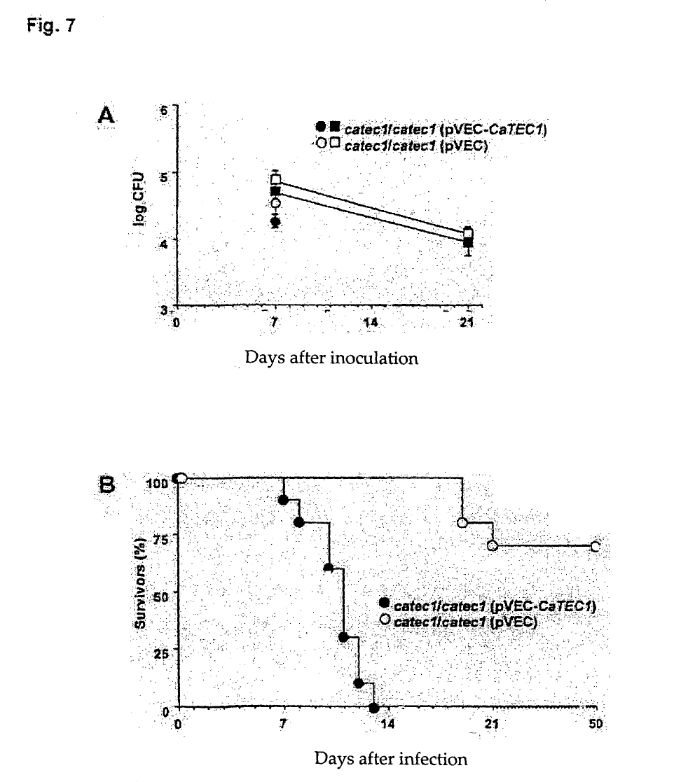

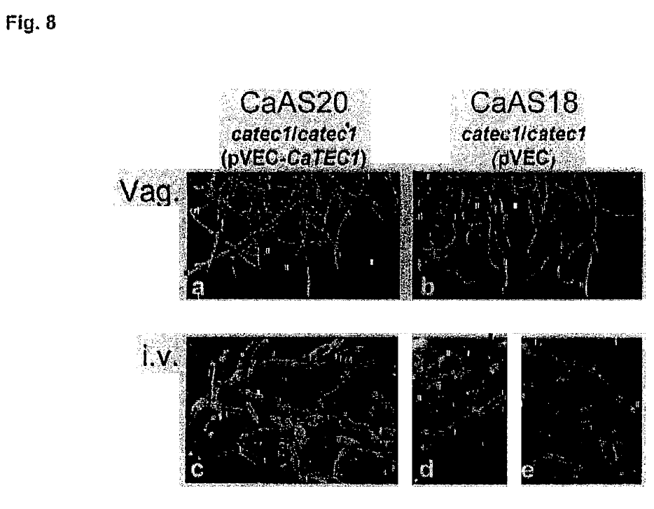

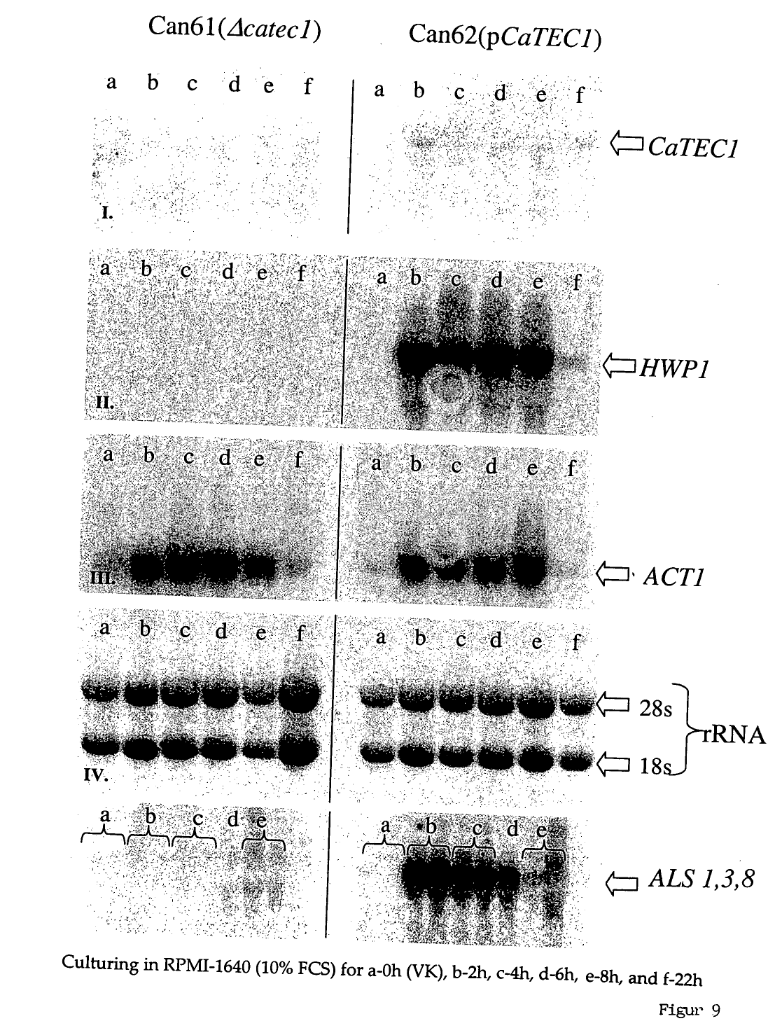

FIG. 6 shows the results of a Northern analysis of SAP4-6 and EFG1 expression in wild type strain SC5314, in CaAS20 and in the mutant CaAS18 during the log growth phase in YPD medium at 28° C. (tracks 1, 5, 9) and after 45 minutes (tracks 2, 6, 10), 90 minutes (tracks 3, 7, 11) and 300 minutes (tracks 4, 8, 12) of growth in liquid culture medium which had been enriched with serum, at 30° C. The ACT1 hybridization as a control is shown in FIGS. 1 and 3.

FIG. 7 shows the results of virulence tests on BALB/c mice which were infected either with the mutant strain CaAS18 CaTEC1/CaTEC1 (pVEC) (open squares or circles) or the revertant CaAS20 CaTEC1/CaTEC1 (pVEC-CaTEC1) (black squares or circles).

(A) shows the fungal load (log CFU) in vaginal flushings of mice after vaginal inoculation with 5×10⁴ C. albicans cells (n=5). The data from two independent experiments are shown.

(B) shows the survival curves of mice (n=10) which had been injected with 5×10⁵ C. albicans cells.

FIG. 8 shows two microscope fluorescent pictures of C. albicans cells which had been purified from KOH-solubilized vaginal flushings (Vag.) and kidneys (i.v.), after staining with Calcofluor White.

Images a and c: revertant CaAS20 (CaTEC1/CaTEC1 pVEC-CaTEC1).

Images b, d and e: mutant CaAS18 (CaTEC1/CaTEC1 pVEC).

FIG. 9 shows the results of Northern blot analyses of RNA of the avirulent mutant Can61 (ΔCaTEC1) (CaAS18) and of the virulent mutant Can 62 (CaAS20) which had been cultured in RPM1-1640 medium containing 10% FCS for different times. The Northern blots were hybridized with probes for the C. albicans specific genes HWP1 and ALS 1, 3, 8. As a control, there followed a hybridization with a CaTEC1 probe and with an ACT1 probe.

### DETAILED DESCRIPTION OF THE INVENTION

The concept “isolated protein” includes proteins which are substantially free from other proteins, nucleic acids, lipids, carbohydrates or other materials with which it is naturally associated. Such proteins include not only recombinant produced protein, but also isolated naturally arising proteins, synthetically produced proteins, or proteins which are produced by a combination of these methods. A recombinant produced variant of CaTEC1 including the secreted protein, can be purified by means of the method described by Smith and Johnson, Gene, 67 (1988), 31-40.

The protein preferably possesses the same properties, particularly the same activity, as the protein which is coded for by a nucleotide sequence with a sequence shown in SEQ ID No. 1 or 2 and whose amino acid sequence is shown in SEQ ID No.4.

The invention furthermore relates to vectors which contain the nucleotide sequences according to the invention. The vectors concerned are preferably plasmids, cosmids, viruses, bacteriophages, shuttle vectors, and other vectors usually used in gene technology. The vectors according to the invention can have further functional units which effect a stabilization and/or replication of the vector in a host organist, or at least contribute thereto.

In a particularly preferred embodiment, the present invention includes vectors in which the nucleotide sequences according to the invention are functionally connected to at least one regulating element. In connection with the present invention, by the concept “regulating element” there are to be understood such elements which ensure the transcription and/or translation of nucleic acid molecules in prokaryotic and/or eukaryotic host cells. Regulating elements can be promoters, enhancers, operators, silencers and/or transcription termination signals. Regulating elements which are functionally connected to a nucleotide sequence according to the invention, in particular to the protein-coding sections of these nucleotide sequences, can be nucleotide sequences which derive from other organisms or other genes than the protein-coding nucleotide sequence itself. Examples of them are: T7, T3, SP6 and further customary regulating elements for in vitro transcription; PLAC, PLtet and further customary regulating elements for expression in E. coli; GAL1-10, MET25; CUP1, ADH1, AFH1, GDH1, TEF1, PMA1 and other regulating elements for expression in S. cerevisiae; GAP1, YPT1, AOX1 and further customary regulating elements for expression in P. pastoris; polyhedrin for expression in Baculovirus systems, and also PCWV, PSV40 and further customary regulating elements for expression in mammalian cells.

The regulating elements used according to the invention can likewise arise from Candida albicans. In particular, the regulating elements can then be the nucleotide sequences according to the invention which code for a protein according to the invention, represented for example in SEQ ID No. 3, which shows the 5′-regulating element including promoter and transcription start site of the CaTEC gene, or in SEQ ID No. 7, which shows the 3′-region of the coding region of the CaTEC1 gene.

In a particularly preferred embodiment, the nucleotide sequence according to the invention or a fragment thereof can be present in the vector both in antisense orientation and also in sense orientation to the regulating element(s). When a nucleotide sequence according to the invention is present in antisense orientation to the regulating element(s), the vector can for example be introduced into a Candida albicans cell and, after the transcription of the nucleotide sequence according to the invention, can inhibit or reduce the expression of the endogenous CaTEC1-GenS/gene of Candida albicans. The fragments present of the nucleotide sequences according to the invention used in the antisense orientation can have a length here which is sufficient to make possible a hybridization to the endogenous CaTEC1 sequences and thus a translation inhibition; for example, a length of at least 100 base pairs. This fragment must of course have a sufficient specificity for the nucleotide sequence to be inhibited.

The vectors according to the invention can furthermore contain further elements. These may be, for example, antibiotics resistance genes, stabilizing elements, and also selection markers or affinity epitopes, for example, HA, Myc, and the like.

The invention of course also comprises vectors which contain not one, but many of the nucleotide sequences according to the invention. These sequences can be arranged such that if necessary one, two, or more of the protein-coding regions according to the invention of the SEQ ID No. 1 or 2 is/are controlled by a single set of regulating elements.

In a preferred embodiment, the present invention relates to host cells which include one or more of the nucleic acid molecules according to the invention or one or more of the vectors according to the invention, and which are capable of expressing the proteins according to the invention. The host cells according to the invention can be either prokaryotic or eukaryotic cells. Examples of prokaryotic cells are bacteria, such as for example Escherichia coli or Bacillus subtilis. Preferred examples of eukaryotic cells according to the invention include yeast cells, plant cells, insect cells and mammalian cells, particularly human cells. The host cells according to the invention can be characterized in that the introduced nucleotide sequence according to the invention is heterologous in relation to the transformed cell, that is, the introduced nucleotide sequence according to the invention does not naturally occur in these cells, or else is localized at a different location or another copy number or orientation in the genome of these cells than is the corresponding naturally occurring sequence.

In an advantageous embodiment of the present invention, the cells concerned are a Gram negative cell, for example, an Escherichia coli cell. In a further advantageous embodiment, however, a Gram positive cell can be concerned, for example Bacillus subtilis. When the nucleotide sequence according to the invention is introduced into a Gram positive cell, the nucleotide sequences according to the invention are preferably connected to a signal peptide which permits a drainage of the translated gene product from the cell into the medium.

In a further preferred embodiment, the host cell according to the invention is a eukaryotic host cell. Here cells can be concerned which already contain one or more such genes on their chromosomes. Eukaryotic cells offer the advantage that the transcription and translation of a nucleotide sequence which codes for a heterologous eukaryotic protein takes place in the same fashion as in the cells from which the eukaryotic nucleotide sequence derives. This means that in eukaryotic host cells the transcripts undergo a correct splicing, and the translation product undergoes the post-translational modifications typical in eukaryotic cells, such as for example glycosylation. In a particularly preferred embodiment, the host cell according to the invention may be a fungal cell, for example a cell of the genus Candida, in particular a Candida albicans cell, a Saccharomyces cell, or an animal cell such as an insect cell. Examples of suitable Candida host cells are derivatives of the strain SC5314 (Fonzi and Irwin, Genetics, 134 (1993), 717-728). Preferred examples of suitable host cells of Saccharomyces cerevisiae are derivatives of the strain S1278b. Further preferred cells include the insect cell line IPLB-Sf21, the human HeLa cell line, Jurkat cells, or CHO cells.

The invention thus also relates to cell cultures which have at least one of the host cells according to the invention, a cell line according to the invention having the capability of producing a protein according to the invention, or a fragment thereof, with the activity for modulating the virulence of Candida albicans cells.

The invention particularly also relates to a method for the production of a virulence-modified protein, particularly a virulence-modified protein from Candida albicans, host cells according to the invention being cultured in a suitable culture medium under such conditions that permit the formation of the virulence-modified protein, and in addition this can be recovered and isolated either from the culture medium or from the cells, at the conclusion of culturing.

In a further preferred embodiment, the present invention relates to an antisense RNA sequence, characterized in that it is complementary to an mRNA which was transcribed from a nucleic acid molecule according to the invention or a portion thereof and can selectively bind to mRNA, this sequence being able to inhibit the synthesis of the CaTEC1 proteins for which the nucleic acid molecules code.

In another preferred embodiment, the present invention relates to a ribozyme, characterized in that it is complementary to a mRNA which was transcribed from a nucleic acid molecule according to the invention or a portion thereof, and can selectively bind to this mRNA and can cleave it, thus being capable of inhibiting the synthesis of the CaTEC1 proteins for which the nucleic acid molecules code. Ribozymes, which consist of a single RNA chain, are RNA enzymes, that is, catalytic RNAs, which can intermolecularly cleave a target RNA. If the strategies described in the literature (cf. e.g. Tanner et al., in: Antisense Research and Applications, CRC Press Inc. (1993), 415-426) are followed, it is possible to construct ribozymes which can cleave the target RNA at a specific place. The two main requirements for such ribozymes are the catalytic domain and regions which are complementary to the target RNA and permit them to bind to their substrate, which is a precondition for cleaving.

The invention also relates, in a further preferred embodiment, to triplex-forming oligonucleotides. These are limited to given target sequences (Barre et al., Nucleic Acids Res. 27 (1999), 743-749). The invention also includes chemical modifications of nucleic acids. Suitable modifications of triplex-forming oligonucleotides are described, for example, in Baba et al., Int J Cancer 72 (1997), 815-820.

Further embodiments of the invention include antisense oligonucleotides. The design of such oligonucleotides, other than that of the triplex-forming oligonucleotides, is based on the selection of a sequence which is complementary to a portion of the mRNA of the transcription factor according to the invention. The design of such oligonucleotides and possible target sequences is described, for example, in Nucleic Acids Res., 26(9) (1998), 2179-2183; Hélène, C., Anti-Cancer Drug Des., 6 (1991), 569-584, and Maher, L. J., Cancer Invest. 14 (1996), 66-82. A screening of different antisense oligonucleotides is often necessary in order to identify the oligonucleotide with the greatest effectiveness. Software-supported methods are also applied to the design of antisense oligonucleotides (Eckstein, F., Nat. Biotechnol., 16 (1998), 24; Matveeva et al., Nat. Biotechnol., 16 (1998), 1374-1375). A method for finding out effective triplex-forming oligonucleotides, antisense nucleotides or ribozymes is described in U.S. Pat. No. 6,013,447.

Oligonucleotides can be chemically modified in order, for example, to increase given properties such as stability and binding strength. Such modifications are likewise a component of the invention. Examples of such modifications which are suitable for antisense oligonucleotides are described in Shchepinov et al., Nucleic Acids Res., 25 (1997), 4447-4454; Tortora et al., Clin. Cancer Res., 5 (1999), 875-881; and Zhou et al., Bioorg. Med. Chem. Lett., 8 (1998), 3269-74.

The invention also includes other structures which can bind to the nucleic acids according to the invention. An example of such structures are peptide nucleic acids (see Kuhn et al, J. Mol. Biol. Mar., 12, 286(5) (1999), 1337-1345, and Nielsen et al., Curr. Opin. Biotechnol., 10 (1999), 71-75).

Further information on the design and application of various oligonucleotides and derivatives thereof is known to one skilled in the art, for example, from Yadava, Molecular Biology Today, 1(1) (2000), 1-16.

The said complementary sequences, that is, the antisense RNA or the ribozyme, are suitable for repression of the CaTEC1 expression, for example in the case of a treatment of a Candida infection. Preferably, the RNA according to the invention and the ribozyme according to the invention are complementary to the coding region of the mRNA, for example, to the 5′-portion of the coding region. One skilled in the art, who has at his disposal the sequences of the nucleic acid molecules according to the invention, can produce and use the above-described antisense RNAs or ribozymes. The region of the antisense RNA or respectively of the ribozyme which was transcribed from the nucleic acid molecules according to the invention preferably has a length of at least 10, in particular of at least 15 and particularly preferably of at least 25 nucleotides.

In a further embodiment, the present invention relates to inhibitors of CaTEC1, which have a similar purpose to the above-mentioned antisense RNAs or ribozymes, that is, the reduction or elimination of biologically active CaTEC1 protein molecules. With such inhibitors, for example, structural analogs of the corresponding protein can be concerned, which act as antagonists. Furthermore such inhibitory molecules are included which are identified using the recombinant produced proteins. For example, the recombinant produced protein can be used in order to screen and identify inhibitors, in that the capability of potential inhibitors to bind to the protein under suitable conditions is used. The inhibitors can for example be identified by producing a text mixture in which the potential inhibitor is incubated with CaTEC1 under suitable conditions, in which preferably CaTEC1 is present in native conformation. Such an in vitro test system can be constructed by methods which are well known to one skilled in the art.

Inhibitors can be identified, for example, in that in a first step, synthetic or naturally occurring molecules are screened which bind to the recombinant produced CaTEC1 protein, and thereafter in a second step, the selected molecules are tested in an in vitro test system or cellular test, with regard to the inhibition of the CaTEC1 protein, which can be detected by means of the inhibition of at least one of the biological activities described hereinafter. Such a screening with regard to molecules which bind CaTEC1 can easily be carried out on a large scale, for example by screening potential molecules capable of binding from substance libraries of synthetic and/or natural molecules. Such an inhibitor can be, for example, a synthetic, organic or chemical substance, a natural fermentation product, a substance extracted from a plant or an animal, or a peptide.

Further examples of inhibitors are specific antibodies. Furthermore, the nucleotide sequence according to the invention and the proteins for which they code can be used for the identification of further factors which take part in the virulence of Candida, for example, a CaTEC1-dependent virulence factor. The proteins according to the invention can, for example, be used for the identification of further (not used) proteins which are connected with the virulence of Candida, screening methods based on protein-protein interactions, for example the two-hybrid system, being used.

The present invention therefore also includes isolated and completely purified monoclonal or polyclonal antibodies or their fragments, which react with a protein according to the invention so specifically and with such affinity that, for example, a detection of the protein according to the invention is possible by means of conventional immunological methods using these monoclonal or polyclonal antibodies or their fragments. A preferred embodiment of the invention therefore includes monoclonal and polyclonal antibodies which can specifically identify and/or bind to a structure of a virulence-modifying protein according to the invention. This structure can be a protein, peptide, carbohydrate, proteoglycan, and/or a lipid complex, which is a portion of the protein according to the invention or has a specific relationship to it. The invention also includes antibodies which are directed against structures which arise as a result of post-translational modifications of the protein according to the invention. The invention also includes fragments of such antibodies such as, for example, Fc- or F(ab′)₂ or respectively Fab fragments.

A further preferred embodiment of the present invention relates to proteins according to the invention or portions thereof, antibodies or portions thereof directed against the proteins, and also nucleotide sequences according to the invention which are present in immobilized form. The nucleotide sequences, proteins and/or antibodies according to the invention, present in immobilized form, can be used as screening devices bound to supports in order to identify substances which enter into reciprocal action with the immobilized molecules. The immobilization can take place to an arbitrary suitable support material, for example to inert or electrically charged, inorganic or organic support materials, such as for example glass materials, aluminum oxide, cellulose, starches, dextran, polyacrylamide, and so on. The support materials used can furthermore have functional groups which for example make possible a covalent binding. An immobilization can take place such that the abovementioned nucleotide sequences, proteins or antibodies are bound into a three-dimensional network.

A further advantageous embodiment of the present invention includes methods for the diagnosis and/or therapy of infections or disease states brought about by Candida species, particularly by Candida albicans, such as Candida mycoses, for example, candidosis of body folds, of the male urethra, the buccal mucosa, or the fingernail or toenail (onchymycosis), of infants' skin, of the vagina, etc., or Candida granuloma. Here the presence of the organisms to be tested is investigated in the presence of the agents according to the invention, particularly nucleic acid molecules, proteins, host cells, vectors, antibodies, or fragments thereof, and their presence is detected. This means that the presence of the agents according to the invention indicates the disease. The present invention therefore also provides a method for the diagnosis of a Candida infection, particularly a Candida albicans infection, including bringing into contact a target sample which is suspected of containing the CaTEC1 protein and/or a nucleic acid coding for CaTEC1, with a reagent which reacts with CaTEC1 and/or a nucleic acid coding for CaTEC1 and the identification of CaTEC1 and/or the nucleic acid coding for CaTEC1. The detection of the said agents according to the invention can take place with corresponding substances, that is, substances specifically detecting the agents according to the invention. For example, a detection of the nucleotide sequences according to the invention can take place by means of hybridized sequences, these preferably being labeled. For example, the hybridized sequences can have fluorescent, enzymatic, or radioactive labels. If the target molecule is thus a nucleic acid, the reagent is typically a CaTEC1-specific nucleic acid probe or a PCR primer. One skilled in the art is able to develop suitable nucleic acid probes based on the CaTEC1 nucleotide sequence. The probes/primer include nucleic acid molecules which hybridize specifically with the nucleic acid molecules according to the invention. The probes/primers are oligonucleotides, which hybridize, preferably under stringent conditions, with at least 10, particularly at least 15, and particularly preferably with at least 25 adjacent nucleotides of the CaTEC1 nucleic acid sequence. The CaTEC1-specific nucleic acid probes/primers preferably include the nucleic acid sequences: 5′-TTT AGG ATC CAA TGA TGT CGC AAG CTA CTC C-3′ and 5′-TTT AGG ATC CAC TAA AAC TCA CTA GTA AAT CCT TCT G-3′. When the target molecule is the CaTEC1 protein, an antibody directed against CaTEC1 is typically used as the reagent. The concept “antibody” relates to antibodies which substantially consist of pooled monoclonal antibodies with different epitope specificities, as well as distinct monoclonal antibody preparations. Monoclonal antibodies are produced with the use of methods well known to one skilled in the art (cf. for example Köhler et al., Nature, 256 (1975), 495), based on an antigen which contains fragments of the CaTEC1 protein according to the invention. The concept “antibody” (Ab) or “monoclonal antibody” (Mab) is to include both intact molecules and also antibody fragments (for example, Fab and F(ab′)₂ fragments) which can bind specifically to the protein. Fab and F(ab′)₂ fragments lack the Fc fragment of an intact antibody. They are more rapidly excluded from the blood circulation and can have a smaller nonspecific tissue binding than an intact antibody (Wahl et al., J. Nucl. Med., 24 (1933), 316-325). The antibodies according to the invention furthermore include chimeras, single-chain and humanized antibodies.

The CaTEC1 protein and/or a nucleic acid coding for CaTEC1, for example in biological liquids or tissues, can be detected directly in situ, for example by an in situ hybridization, or can be isolated from other cellular components using methods which are known to one skilled in the art before bringing into contact with a probe. Detection methods include Northern blot analysis, RNase protection tests, in situ methods, for example in situ hybridization, in vitro amplification methods such as PCR, RT-PCR, LCR, QRNA replicase, or RNA transcription/amplification, reverse dot blot methods, such as that disclosed in EP-B1 0 237 362, immunoassays, Western blot methods, and other detection tests. Products which are obtained by the use of in vitro amplification methods can be detected by known methods, for example by the separation of the products on agarose gel and subsequent staining with ethidium bromide. In another embodiment, the amplified products can be detected by the use of labeled amplification primers or labeled dNTPs. The probes/primers according to the invention can be detected by labeling with a radioisotope, a bioluminescent compound, a chemiluminescent compound, a fluorescent compound, a metal chelate, or an enzyme.

The detection of proteins or antibodies according to the invention preferably takes place using antibodies which are directed against the said proteins.

Furthermore, the expression of CaTEC1 in tissues (in the case of a systemic infection) with conventional immunohistological methods (Jalkanen et al., J. Cell. Biol., 101 (1985), 976-985; Jalkanen et al., J. Cell. Biol., 105 (1987), 3087-3096; Sobol et al., Clin. Immunopathol., 24 (1982), 139-144; Sobol et al., Cancer, 65 (1985), 2005-2010). Other methods based on antibodies which are suitable for the detection of gene expression include immune tests such as the enzyme-linked immunosorbent assay (ELISA) and radioimmunoassays (RIA). Suitable labels are known to one skilled in the art and include enzyme labels such as glucose oxidase, and radioisotopes such a iodine (¹²⁵I, ¹²¹I), carbon (¹⁴C), sulfur (³⁵S), tritium (³H), indium (¹¹²IN) and technetium (⁹⁹mTc), and fluorescent labels such as fluorescein and rhodamine, and also biotin. The protein can also be detected in vivo by means of image-forming methods. Antibody labels or labels for imagewise display of the protein include those which can be detected using X-ray radiography, NMR or ESR. Labels suitable for X-ray radiography include radioisotopes such as barium or cesium, which emit a suitable radiation, but which are not injurious to a subject. Labels suitable for NMR and ESR methods include those with a detectable characteristic spin, for example deuterium. A protein-specific antibody or antibody fragment which is detectable with a suitable detectable unit for imagewise display, such as a radioisotope, for example ¹³¹I, ¹¹²In, ⁹⁹mTc, a substance opaque to X-rays, or a material which can be detected by nuclear magnetic resonance methods, is introduced, for example, parenterally, subcutaneously, or intraperitoneally into a mammal. The size of the individual and the imaging system used determine the amount of the unit used for imagewise display, which is required for imagewise displays suitable for diagnosis. In the case of a radioisotope unit, the amount of the injected radioactivity for a human subject is usually in the range of about 5-20 millicuries of ⁹⁹mTc. The labeled antibody or the labeled antibody fragment is then preferably concentrated at the places of a cell which contain the specific protein.

The present invention therefore also relates to the use as diagnostic agents or the nucleic acid molecules, proteins, host cells, vectors, antibodies and/or portions thereof, according to the invention. In connection with the present invention, “diagnostic agents” is to be understood to mean any material which can specifically detect the presence of states, processes or substances, or respectively their absence, and in particular can give conclusions regarding diseases. Diagnostic agents frequently have detecting and labeling functions.

It goes without saying that the invention also includes diagnostic kits which contain the agents, that is, nucleotide sequences, proteins, antibodies or portions thereof, and which permit the diagnosis of diseases brought about by Candida species, particularly Candida albicans. In connection with the present invention, there are to be understood as diseases, in particular also states such as unnatural emotional states, signs of old age, disturbances of development, and the like.

The invention also includes methods for the therapy of diseases brought about by Candida species, particularly Candida albicans, preferably a systemic infection, the organism to be treated being treated with the agents according to the invention for a period and with a dose which is sufficient to stabilize or improve the disease picture or to cure the disease. The method according to the invention for the treatment of a Candida infection therefore includes the administration of a therapeutically effective amount of a reagent which diminishes or inhibits CaTEC1 activity. Examples of such reagents are the previously described antisense RNAs, ribozymes or inhibitors, for example an antibody directed against CaTEC1. The inventing therefore also relates to the use of the nucleic acid molecules, proteins, host cells, vectors, inhibitors, antisense RNAs, ribozymes, antibodies and fragments thereof as therapeutic agents. In connection with the present invention, under the concept “therapeutic agents” are to be understood those materials which are either used prophylactically or in the course of a disease in order to prevent, alleviate or eliminate disease states. Vaccines also belong to these, for example. In connection with the present invention, there are also understood under “therapeutic agents” those materials which exclusively or also have cosmetic effects.

The present invention also relates to a pharmaceutical composition which contains a reagent which diminishes or inhibits the CaTEC1 expression or the activity of the CaTEC1 protein. For example, the administration of an antibody which is directed against the protein can also reduce or eliminate the activity of the protein.

In a preferred embodiment, the invention therefore also relates to pharmaceutical compositions which contain the agents according to the invention, thus the nucleic acid molecules, proteins, host cells, vectors, inhibitors, antisense RNAs, ribozymes, antibodies, or fragments thereof, according to the invention, if necessary together with a pharmaceutically compatible carrier, and if necessary further additives such as stabilizers, thickeners, separating agents, lubricants, colorants, odorants, flavorings, emulsifiers, or the like.

For administration, these agents can preferably be combined with suitable pharmaceutical carriers. Examples of suitable pharmaceutical carriers are well known to one skilled in the art and include phosphate-buffered physiological saline solutions, water, emulsions such as oil-in-water emulsions, various types of wetting agents, sterile solutions, etc. Such carriers can be formulated by conventional methods and can be administered to the patient in a suitable dose. The administration of suitable compositions can take place in various ways, for example, by intravenous, intraperitoneal, subcutaneous, intramuscular, topical, or intradermal administration. The administration of course depends on the kind of Candida infection (for example, systemic or vaginal infection) and on the kind of compound which is contained in the pharmaceutical composition. As is well known, the dosage for a given patient depends on many factors, to which belong the age and sex of the patient, the specific compound to be administered, the period and path of administration, the kind and seat of the infection, the general state of health and the simultaneous administration of other medicaments.

The antisense RNAs or ribozymes according to the invention can be used directly or are preferably administered with the use of a recombinant expression vector, for example, a chimeric virus which contains these compounds, or a colloidal dispersion system. By the delivery of these nucleic acids to the desired target, the intracellular expression of CaTEC1 and therefore also the amount of the CaTEC1 protein can be diminished, which leads to the inhibition of the virulence of Candida.

A direct application to the target place can for example take place by means of a ballistic delivery system, such as a colloidal dispersion system or by means of a catheter into an artery. The colloidal dispersion systems which can be used for the delivery of the abovementioned nucleic acids include macromolecular complexes, nanocapsules, microspheres, beads, and systems based on lipids including oil-in-water emulsions, (mixed) micelles, liposomes and lipoplexes. A preferred colloidal system is a liposome. The liposome is normally composed of phospholipids and steroids, particularly cholesterol. One skilled in the art can select such liposomes which are suitable for the delivery of the desired nucleic acid molecule. In order to attain delivery at only the desired plate, for example a tumor, organ-specific or cell-specific liposomes can be used. Using conventional methods, one skilled in the art can produce liposomes which are specifically directed to a target. Such targeting methods include passive targeting, using the natural tendency of liposomes to distribute in cells of the reticuloendothelial system (RES) in organs which contain sinusoid capillaries, or an active targeting, for example by coupling of the liposomes to a specific ligand, for example, an antibody, a receptor, a sugar, a glycolipid, a protein, etc., with the application of well known methods. Monoclonal antibodies are preferably used according to the invention in order to deliver a correctly targeted supply of liposomes to specific organs or tissues by means of specific cell surface ligands.

Preferred recombinant vectors which are suitable for gene therapy (particularly in the case of a systemic infection) are viral vectors, for example, vectors based on adenoviruses, herpes viruses, vaccinia viruses or preferably those based on RNA viruses, such as a retrovirus. The retroviral vector is preferably a derivative of a retrovirus coming from rodents or birds. Examples of such retroviral vectors which can be used in the present invention are: the Moloney leukemia virus of rodents (MoMuLV), the Harvey sarcoma virus of rodents (HaMuSV), the rodent mamma tumor virus (MuMTV), and the Rous sarcoma virus (RSV). Preferably a retroviral vector from a non-human primate is used, such as the gibbon leukemia virus (GaLV), which has a wider host range than the murine vectors. Since recombinant retroviruses have defects, helper cells are for example necessary so that infectious particles can be produced. For example, helper cell lines can be used which contain plasmids which code for all the structural genes of the retrovirus under the control of regulating sequences within the LTR region. Suitable helper cell lines are well known to one skilled in the art. The vectors can additionally contain a gene which codes for a selectable marker so that transduced cells can be identified. Furthermore, the retroviral vectors can be modified so that they are target-specific. This can for example be attained by insertion of a polynucleotide region which codes for a sugar, a glycolipid, or a protein, preferably an antibody. One skilled in the art knows further methods for the production of target-specific vectors. Further suitable vectors and methods are described in the literature and are known to one skilled in the art (cf. e.g. WO 94/29469 or WO 97/00957).

The present invention also provides kits which can be used in diagnostic research. Such kits are suitable for the detection of Candida, based on the CaTEC1 protein or on a nucleic acid coding for CaTEC1. A kit according to the invention includes a probe for the detection of the CaTEC1 protein and/or of a nucleic acid coding for CaTEC1. For detection, the probe can be labeled. The probe is preferably a specific antibody or a specific nucleic acid probe, or respectively, primer. In a preferred embodiment, the kit contains a monoclonal antibody against CaTEC1 and makes the diagnosis possible, for example by means of ELISA, and contains the antibody bound to a solid support, for example, a polystyrene microtiter plate or nitrocellulose; methods from the known field of art can be used. In another embodiment, the kits are based on a RIA test and contain an antibody which is labeled with a radioactive isotope. In a preferred embodiment of the kit according to the invention, the antibody is labeled with enzymes, fluorescent compounds, luminescent compounds, ferromagnetic probes, or radioactive compounds. The kit according to the invention can also include one or more containers which, for example, contain one or more probes according to the invention.

A particularly preferred embodiment of the present invention relates to methods for screening, that is, for finding out and identifying substances which are suitable for the treatment of diseases brought about by Candida species. Here the substances to be tested for their therapeutic effect are brought into contact in a suitable medium, such as for example a solution, suspension, or with a cell, with an agent according to the invention, particularly a nucleotide sequence according to the invention or a protein or fragment thereof according to the invention, and a reciprocal action which possibly takes place, for example binding, is detected.

A preferred substance screening method of the present invention comprises an assay which is constructed as follows: The promoters of the isogene of the secreted aspartyl proteinase of type 4, 5 and 6 (SAP5-6 gene) contain a head-to-tail arrangement, intersected by separating elements, or TCS motifs which represent the binding sites for fungal TEA/ATTS transcription factors, particularly the CaTEC1 factor according to the invention. Since it could be shown according to the invention that the CaTEC1 protein can induce the expression of the SAP4-6 gene by binding to the promoter, the promoter region of this SAP4-6 gene is combined with a reporter gene. The expression of the reporter gene can be induced either by the addition of the purified CaTEC1 protein or by the expression of the nucleotide sequences according to the invention coding for the CaTEC1 protein, the CaTEC1 protein binding under suitable conditions to the promoter region and inducing the expression of the reporter gene. The inducibility of the system used in the test system for the expression of the reporter gene is then tested in the absence or presence of the substances to be tested. By means of libraries of natural materials and substances, suitable inhibitors can thus be detected which inhibit the function of the CaTEC1 protein or the expression of the nucleotide sequences according to the invention which code for the CaTEC1 protein. In this manner, substances can thus be identified which represent potential medicaments for combating Candida infections.

Likewise, the proteins according to the invention can be used in the form of hybrid proteins in conventional two-hybrid systems, in order to identify the proteins coming into reciprocal action with the proteins according to the invention, a first hybrid protein and for example a domain transactivating the transcription of a reporter gene being produced and incubated together with a reporter gene and a second hybrid protein from a first fraction which represents the protein to be investigated and a second fraction which represents a DNA binding domain of the transcription activating fraction of the first hybrid protein. With interactions of the protein to be investigated with the protein according to the invention, there now occurs an association of the transactivating and the DNA binding domains, which furthermore induces the expression of the reporter gene and permits the detection of the binding. Optional other combinations are of course possible.

The proteins according to the invention are therefore effective means for screening substances of pharmacological interest for the treatment of diseases brought about by Candida species. The proteins according to the invention can be used in isolated form, in medical devices, in drug delivery devices, matrices for drug screening libraries, microtiter plates, applicable catalysis devices, or the like.

A further preferred embodiment of the present invention relates to a mutated Candida albicans cell, characterized in that in it the nucleotide sequences according to the invention or the regulating regions are altered such that no transcript of the nucleotide sequence according to the invention can be detected. The Candida albicans cell modified according to the invention can either be a haploid cell, the nucleotide sequence according to the invention present in only one copy being altered or mutated, or a diploid cell in which both copies of the nucleotide sequence according to the invention are altered or mutated such that a homozygotic null mutant is concerned. Here both a natural cell mutant and a mutant produced by technical means, for example a mutant produced by means of homologous recombinations, antisense methods, or the like, can be concerned. In a particularly preferred embodiment, the present invention relates to homozygote ura3/ura3 catec1/catec1 (pVEC) null mutant CaAS18, which was produced by sequential homologous recombination and was deposited at the DSMZ in Braunschweig under the number DSM 13722 on Sep. 6, 2000.

The mutated Candida albicans cell according to the invention is characterized in that its viability, colony size and doubling time is not, or only insignificantly, altered in comparison with wild-type cells. A mutated Candida albicans cell according to the invention is in particular characterized in that its filamentous growth is impaired, in particular the formation of germ tubes and true hyphae being suppressed. Instead of this, mutated cells according to the invention form curved tubular structures which have constrictions within the cell-cell boundaries and thus represent morphological pseudohyphae. Besides the morphological changes, such mutated Candida albicans cells are characterized in that the expression of the SAP4-6 gene is not inducible in them. In a systemic infection in a mouse model of systemic Candida mycoses, it is found that the virulence of the homozygotic catec1 cell mutants of Candida albicans is significantly suppressed in comparison with wild-type cells.

A preferred embodiment of the present invention therefore relates to the use of living mutated or altered Candida albicans cells, particularly the catec1/catec1 null mutant CaAS18, with a mutation in the nucleotide sequences according to the invention, so that these nucleotide sequences can produce no transcript under transcription conditions, and the cells show a considerably reduced virulence as against wild-type cells, as an inoculant or vaccine in the context of a protective inoculation for producing a resilient disease immunity against infections of Candida wild-type species, particularly of Candida albicans. Here the live vaccines are preferably attenuated with further suitable means so that, for example, their ability to proliferate is eliminated. The administration of such an attenuated living vaccine can, for example, take place parenterally, locally, particularly orally, nasally, cutaneously, or by means of inhalation. Administration pursues the target of, in particular, a local infective attack on mucous membranes by the formation of secretory antibodies and by increasing the macrophage activity.

The sequence protocol belonging to this teaching includes:

SEQ ID No. 1 shows the DNA sequence comprising 4216 nucleotides of a genomic clone of the CaTEC1 gene from Candida albicans.

SEQ ID No. 2 shows the protein-coding region, comprising 2229 nucleotides, of the genomic clone from SEQ ID No. 1 (by 953-3181, relative to SEQ ID No. 1 ATG start: 953-955, stop codon: 3182-3184).

SEQ ID No. 3 shows the 5-regulating element of the CaTEC1 gene of SEQ ID No. 1 (bp 1-952 relative to SEQ ID No. 1).

SEQ ID No. 4 shows the amino acid sequence of the CaTEC1 protein according to the invention, comprising 743 amino acids, derived from an open reading frame (SEQ ID No. 2) of SEQ ID No. 1.

SEQ ID No. 5 shows the sequence of the primer TEC1 gene PCR.01 which was used for the isolation of the DNA sequence according to the invention of the CaTEC1 gene.

SEQ ID No. 6 shows the sequence of the primer TEC1 gene PCR.02 which was used for the isolation of the DNA sequence according to the invention of the CaTEC1 gene.

SEQ ID No. 7 shows the 3′-regulating element of the CaTEC1 protein according to the invention of SEQ ID No. 1 (bp 3185-4216 relative to SEQ ID No. 1).

SEQ ID No. 8 shows the sequence of a CaTEC1 specific nucleic acid probe suitable for the diagnosis of Candida infections.

SEQ ID No. 9 shows the sequence of a further nucleic acid probe suitable for the diagnosis of Candida infections.

### EXAMPLE 1

### Cloning and Expression of the CaTEC1 Gene

For the amplification of the CaTEC1 gene by means of the PCR method, there were used the primer TEC1genePCR.01 with the sequence:

5′-TTTTCTATTCTAACCACCCTCTGC-3′ (SEQ ID No. 5)

and the primer TEC1genePCR.02 with the sequence:

5′-CCCGCCTTGCCCCTCTT-3′ (SEQ ID No. 6).

By means of these primers, a 4.2 kb fragment of genomic DNA of the strain CA1-4 (Fonzi and Irwin, Genetics (1993) 134, 717-728) was recovered using the LongRange PCR Kit (Boehringer, Germany). The PCR product was subcloned in the EcoRV site of the vector pGEM-T-Easy (Promega, Germany), the plasmid p275 deposited at the DSMZ being obtained. The NotI fragment of the plasmid p275 which contained the CaTEC1 sequence was cloned in the NotI site of the vector pRS425 (Christianson et al., Gene, 110 (1992), 119-122), the vector pRS425CaTEC1 for complementation analysis in S. cerevisiae being obtained.

The BgIII-EcoRV fragment of the plasmid p275 which contained the CaTEC1-ORF (open reading frame, 2229 bp long, SEQ ID No. 2) was exchanged for the 3.5 kb BgIII-SaII fragment of a hisG-URA3-hisG cassette which was obtained from the plasmid pMB7 (Fonzi and Irwin, Genetics, 134 (1993), 717-728), the plasmid p277 being obtained. This plasmid was cleaved with NotI and transformed into the ura⁻ C. albicans strain CA1-4, in order to thus substitute the hisG-URA3-hisG cassette for the coding region of one of the chromosomal CaTEC1 alleles by means of homologous recombination. Ura+transformants were selected on a selective ura⁻medium. The integration of the cassette in the CaTEC1 site was confirmed by the Southern blot analysis. Spontaneously arising ura⁻ derivatives were selected on a medium which contained 5-fluoroorotic acid. These clones were inspected by means of Southern blot hybridization in order to identify the clone which had lost the URA3 gene by means of intrachromosomal recombination which had been brought about by the hisG repetition sequences. This process was repeated in order to delete the other functional allele of CaTEC1. The homozygotic catec1::hisG/catec1::hisG mutant (one of these homozygotic mutants was designated as CaAS15) was thereby obtained. The mutant CaAS15 was either transformed with the vector pVEC, the strain CaAS18 being obtained, or with the vector pVEC-CaTEC1, which contained the CaTEC1 gene, the CaAS20 strain being obtained.

The C. albicans plasmid pVEC-CaTEC1 was constructed in that a NcoI-SacI fragment of the plasmid p275 which contains the CaTEC1 gene was cloned in the plasmid pVEC (Candida albicans, Navarro-Garcia et al., J. Med. Vet. Mycol 33 (1995), 361-366), which contains a replication starting point of Candida albicans and selectable CaURA3 marker and which was cleaved with the restriction enzymes SmaI and SacI.

### EXAMPLE 2

### Function Analyses in S. cerevisiae

Yeast tec1 mutants have a pseudohyphal defect in diploid and an invasion defect in haploid strains. CaTEC1 was introduced into the diploid S. cerevisiae strain L6146 (tec1/tec1). L6146-pRS425CaTEC1 was obtained. This strain was tested for pseudohyphal growth on SLAD-agar. It could be shown that CaTEC1 could complement the pseudohyphal growth defect of L6146, where the strain L6146 contained the plasmid without CaTEC1 insert. Likewise, the invasion growth defect of the haploid S. cerevisiae strain L6149 (tec1) on YPD agar could by complemented after transformation with pRS425CaTEC1. It could be shown that in the presence of CaTEC1 not only haploid (MATa) bur also diploid (MATa/α) mutants could be induced to invasion into the surface of the agar. This could be attributed to an over-activity of the TEC1-reactive elements in S. cerevisiae due to high CaTEC1 copy numbers. CaTEC1 is capable of activating the FL011 transcription in S. cerevisiae in the absence of TEC1. F1011p namely requires Tec1p for activation and is essential for the pseudohyphal- and invasive growth (Lambrechts et al., Proc. Natl. Acad. Sci. USA (1996) 93, 8419-8424). In the presence of CaTEC1, a FL011-lacZ reporter (Rupp et al., EMBO J. (1999) 18, 1257-1269) was induced 7-fold or 4-fold, both in diploid (L6146 tec1/tec1 pRS425CaTEC1) and in haploid (L6149 tec1 pRS425CaTEC1) S. cerevisiae strains. In the presence of CaTEC1, a FG::TyA-lacZ reporter was not activated, which indicated that an interaction with Stec12p did not occur, which would be necessary to activate FRE elements.

### EXAMPLE 3

### Function Analyses in C. albicans

A sequential homologous recombination (Fonzi and Irwin, (1993), loc. cit.) was carried out in order to delete the two CaTEC1 alleles in C. albicans and to obtain the homozygotic ura3/ura3 catec1/catec1 null mutant CaAS15. The obtained genotypes were confirmed by Southern blot analysis. Northern blots showed that the CaTEC1 transcript was not present in the strain CaAS18, which was obtained by transformation of an empty pVEC plasmid (Navarro-Garcia et al. (1995), loc. cit.) into CaAS15. CaTEC1 mRNA was detected in the strain CaAS20 which was obtained by transformation of the plasmid pVEC-CaTEC1 into CaAS15 in amounts which correspond to those in wild type strains.

The survivability, colony size and generation time of C. albicans cells in vitro was not essentially affected by a deletion of one or both CaTEC1 alleles. The deletion of both CaTEC1 alleles, however, negatively affected the filamentary growth in vitro. When hyphae formation in liquid medium was induced by serum at 37° C. for 1.5 or 3 hours, it could be observed that the mutant CaAS18 had a suppressed formation of germ tubes and true hyphae. Instead of these, these cells formed curved, tubular structures with constrictions at the cell-cell boundaries, which indicate a pseudohyphal morphology.

The isogenous catec1/catec1 mutants could not be excited to hypha formation either by serum induction or by interaction with murine phagocytes.

Most of the mutant cells formed short filaments 24 hours after induction. A smaller fraction of the cells developed into branched mycelia. The same morphology of the CaAS18 strain was obtained by the induction of hyphal growth in modified Lee's medium at neutral pH. These defects in liquid medium could be reverted by the reintroduction of the CaTEC1 gene on the plasmid pVEC-CaTEC1 in the revertant strain CaAS20.

The interaction of the mutant CaAS18 and the revertant CaAS20 cells with inflammatory phagocytes in vitro was likewise investigated. During an 8-hour interaction with MΦ, CaAS20 germ tubes extended into hyphal cells, which obviously serve for C. albicans to be able to escape by growing out from the effects of phagocytes. CaAS18 was not able to escape MΦ by growing out, probably due to the suppressed formation of extended and filamentous cells. Clusters of CaAS18 were co-localized with MΦ, probably due to phagocytosis by MΦ. After 24 hours, the number of C. albicans cells in the CaAS18-infected MΦ was increased from 1.8 to 3.9 C. albicans cells per infected MΦ. This indicates that CaAS18 constantly grows within the host cells. Occasionally a few mycelia could be observed in a small number. Most of the cells of the strain CaAS20 developed into typical mycelia. Hyphal growth was not inhibited by MΦ. CaTEC1p is therefore necessary for the growing out of C. albicans from MΦ.

Moreover, an expression of the supposed target gene SAP4-6 on CaAS18 mutants could not be induced, while the wild type strain SC5314 and the revertant strain CaAS20 produced SAP4-6 mRNA in comparable amounts. A different result was obtained when a EFG-1 probe was used for a Northern hybridization of the same RNA probes. All three isogenous strains expressed the EFG-1 gene. This shows that Tec1p is not required for EFG-1 transcription. Moreover, the wild type expression of Efg1p is not sufficient to ensure a normal, hyphal growth in the absence of CaTEC1p, which indicates further functions of CaTec1p in hypha formation.

### EXAMPLE 4

### Virulence Analyses in C. albicans

To investigate the significance of CaTEC1 for the virulence of C. albicans, mucosal and systemic models of murine candidosis were used. BALB/c mice were inoculated intravaginally or intravenously with CaAS18 and CaAS20 C. albicans cells. The replication of the fungus on the vaginal mucosa and the survival rate were investigated. The deletion of both alleles of CaTEC1 had no significant effect on the number of C. albicans colony forming units (CFU) taken from the vaginal region: colonization with both CaAS20 and also with CaAS18 led to comparable CFU values on days 7 and 21. A more precise microscopic investigation with Calcofluor White stained C. albicans cells from vaginal flushings showed that both strains grew in comparable filamentous form. Obviously C. albicans is capable, in the absence of CaTEC1, to survive and propagate on mouse vaginal mucosa.

In a mouse model of systemic candidosis (Csank et al., Mol Biol Cell (1997) 8, 2539-2547, Csank et al., Infect Immun (1998) 66, 2713-2721, Timpel et al., J. Bacteriol (2000) 182, 3063-3071), the inoculation with CAS20 cells led to 100% mortality after 13 days. In contrast to this, 70% of the mice infected with the mutant CaAS18 cells survived at least 50 days, and the surviving animals showed no clinical symptoms. A statistical investigation using the log rank test of the survival curves showed very high statistical significance (p<0.0001). 100×10⁵ CaAS18 cells had to be injected in order to produce a clinical course comparable to that observed with 5×10⁵ CaAS20 cells.

The gene defect in the CaTEC1 gene thus has serious consequences, not only for hypha formation in vitro, but also for the virulence of C. albicans in vivo. A catec1/catec1 mutant in infected Balb/c mice brings about only moderate clinical signs of a systemic candidosis at an infection dose of 5×10⁵ cells. In contrast to this, an isogenous wild type control strain brings about 100% mortality already after 15 days (average survival time=10 days).

A comparison of the morphology of mutant and revertant cells which originated from the kidneys of infected animals and were stained with Calcofluor White showed that CaAS20 and CaAS18 have the same potential for hyphae formation in vivo, that is, signal of the host given off by C. albicans during the mucosal or systemic infection induce filamentous growth in the absence of CaTEC1. Surprisingly, the virulence is significantly suppressed in catec1/catec1 mutants during systemic infection, although hypha formation is observable. This shows that CaTec1p is important for the systemic C. albicans infection, since it regulates a virulence mechanism by means of a mechanism which follows the morphological conversion of the yeast form into the hyphal form.

The results show that the gene CaTEC1 according to the invention regulates both hypha formation and also virulence of C. albicans. The virulence appears to be regulated on two levels by CaTec1p. On the one hand, the expression of the type 4, 5 and 6 isogenes of the secreted aspartyl proteinases (SAP 4-6) is regulated, sap4-6 triple mutants appearing to be avirulent in vivo (Monod et al., Mol. Microbiol., 13 (1994), 357-368). Secondly, CaTec1p regulates hypha formation in vitro and is necessary for the rapid growth of C. albicans before MΦ.

The experiments described allow CaTec1p to appear as a target molecule of interest for the development of new antimycotics, particularly with regard to the development of systemic and topical fungicidal chemotherapies.

### EXAMPLE 5

### Investigation of the Gene Regulated by CaTEC1

The Candida albicans strains Can61 (ΔCaTEC1) and Can62 (pCaTEC1) were taken from the collection of strains, plated on SC-ura-agar, and incubated for two days in the incubator at 30° C. Thereafter respective 5 ml pre-cultures were inoculated into SC-ura liquid medium from the grown colonies, and were incubated overnight in shaking incubators at 30° C. and 37° C. From these pre-cultures, the main cultures were set up in a 1:100 ratio in respectively 5 ml YPD medium, RPMI-1640 medium containing 10% FCS and α-Mem medium, and were again shaken at 30° C. or 37° C. respectively. In the main trial, as in the preliminary trial, plating of the strains Can61 (ΔCaTEC1) and Can62 (pCaTEC1) and also of the wild type Can14 was performed on SC-ura-agar plates, which were kept at 30° C. for 2 days in the incubator. Differing from the preliminary trial, respectively 50 ml of SC-ura liquid medium was inoculated as a pre-culture and shaken in the shaking incubator overnight, exclusively at 30° C. The main culture of the strains Can61 (ΔCaTEC1) and Can62 (pCaTEC1) took place on a 1:100 scale from the corresponding pre-culture in respectively 600 ml RMI-1,640 [sic] medium with an addition of 10% FCS. Culture took place over a period of 2-16 hours and also overnight, likewise at 30° C. in a shaking incubator. In addition, 50 ml of YPD medium was inoculated in a 1:100 ratio with an overnight culture of the wild type Can14 similarly grown in SC-ura. This culture took place exclusively overnight at 30° C. in the shaking incubator. From the strains thus cultured, total RNA was isolated as described hereinafter. After LiCl precipitation, the isolated RNA concentration was determined. The isolated RNA was subjected to gel electrophoresis and then transferred to a nylon membrane for carrying out a Northern blot analysis.

Hybridization with DNA probes for the genes CaTEC1, HWP1, ACT1 and ALS 1, 3, 8 was then performed. The results of this Northern blot analysis are shown in FIG. 9. As can be seen in FIG. 9, the gene HWP1 was not expressed in the avirulent mutant Can61, in which the CaTEC1 is deleted, even after 22 hours of incubation. In contrast to this, HWP1 was already expressed in the virulent mutant 62 after 2 hours of culturing, the level of expression not being increased even after longer culturing. Also the genes ALS 1, 3, 8 were not expressed in the avirulent mutant Can61 (ΔCaTEC1). In the virulent mutant Can62 (CaTEC1), the expression of ALS 1, 3, 8 is highest at 4 hours of culturing, and decreases during further culturing. As a control, the expression of the actin gene was tested in both mutants, and it was shown that ACT1 is already expressed after two hours in both the avirulent and in the virulent mutants. The results show that the expression of the genes HWP1 and ALS 1, 3, 8 is under the control of the transcription factor CaTEC1, in contrast to which the expression of the actin gene is not regulated by CaTEC1.

### EXAMPLE 6

### Investigation of Genes Regulated by CaTEC1, Using a Chip

As described hereinafter, a chip was produced containing nucleic acids of Candida albicans which either have homologies to cell wall genes of Saccharomyces cerevisiae, or which were known cell wall genes of Candida albicans. These probes were amplified by means of PCR on a 96 well plate scale and were then purified. Thereafter the probes were printed onto the coated slides.

25 μg of two RNAs to be compared on a chip were subjected to a reverse transcription with Superscript II as described hereinafter, in which respectively different fluorescent labeled nucleotides (Cy3-dCTP and Cy5-dCTP) were incorporated into the existing cDNA. Unincorporated nucleotides were separated using a Microcon-30 filter. After purification of both individual samples, concentration took place. Large particles were then eliminated using a Millipore filter (0.45 μm).

Hybridization then took place. This was carried out as follows. After a petri dish had been prepared with moist Whatman paper as a hybridization chamber and the sample to be applied had been denatured at close to 100° C., the sample was applied between the markings on the DNA chip. Air bubbles then arising could be destroyed with a hot needle. A cover glass was then put in place, free from air bubbles, and the chamber was carefully closed with Parafilm. Hybridization took place overnight in a 65° C. water bath, the applied fluorescent labeled cDNA then being able to reciprocally act and form hydrogen bonds with the complementary DNA on the chip.

Sample not bound to the chip by hybridization was removed using three washing steps of respectively lower salt concentration. After subsequent centrifuging at 150×g for 5 minutes, the chips were dry and could be scanned in the array scanner.

The samples bound to the slide could be detected by means of a special array scanner. Here light of different wavelengths was emitted by two lasers and excited the two different fluorescent dyes. As a result, the scanner supplied two individual images of the same array, once in red light and once in green, with the probes respectively fluorescing to which hybridization the samples occurred. A so-called overlay could be produced therefrom by means of the ImaGene software. Both individual images, which had been superposed by the computers, thus provided an image in which the same gene expressions appear as yellow data points, while different gene expressions appear as either red or green data points. This imagewise representation of the results was expanded by a further evaluation of the raw data table supplied by the computer. Here its own fluorescence intensity and the related background intensity was shown for each fluorescing data point on the array. The computer determined these based on the pixels which were calculated over a surface laid around the data point. Based on this table, a further evaluation was possible by means of conventional software, for example Excel. From the evaluations, results were obtained which confirm the results of the Northern blot analysis.

### EXAMPLE 7

### Materials and Methods

1. Materials

| 1.1 Biological materials | |
|---|---|
| Can14: | Wild type, clinical isolate SC5341 [6] |
| Can 61 (Δcatec1): | avirulent mutant CaAS18 [26] |
| | ura3 : : imm434/ura3 : : imm434 |
| | catec1 : : hisG/catec1 : : hisG |
| | plasmid pVEC (ARS-URA3) |
| Can 62 (pCaTEC1): | virulent mutant CaAS20 [26] |
| | ura3 : : imm434/ura3 : : imm434 |
| | catec1 : : hisG/catec1 : : hisG |
| | plasmid pVEC-CaTEC1 (ARS-URA3) |

1.2 Chemicals, Buffers and Solutions

If not otherwise stated, the chemicals used were of analytical grade from Carl Roth GmbH of Karlsruhe; the water was taken from a Millipore plant (ddH₂O). All solutions used for RNA isolation were prepared with DEPC-H₂O.

(a) Related Buffers and Solutions

| | |
|---|---|
| Aldrich: | N-methyl-2-pyrrolidinone, water-free |
| | 99.5% succinic acid anhydride 99+% |
| Eppendorf: | Microbiologically pure water |
| Gibco Life Technologies: | PBS (10×), pH 7.4 |
| Sigma Diagnostics: | Poly-L-lysine solution |

(b) Buffers and Solutions Used

| | |
|---|---|
| Church buffer: | 7% (W/v) SDS |
| | 1% (W/v) BSA |
| | 1 mM EDTA |
| | 250 mM sodium phosphate buffer |
| | pH 7.2 |
| DEPC-H₂O | 1 ml DEPC in 1 l ddH₂O |
| | Autoclaved |
| DNA loading buffer (6×) | 0.25% bromophenol blue |
| | 0.25% xylene cyanol FF |
| | 30% glycerol in water |
| MOPS (10×) | 0.4 M MOPS pH 7.0 |
| | 0.1 M sodium acetate |
| | 0.5 M EDTA pH 8.0 |
| Sodium borate | 1 M boric acid, adjust to pH 8.0 with NaOH |
| Phenol | dissolve solid phenol in ddH₂O until saturated |
| RNA loading buffer | 50% glycerol |
| | 1 mM EDTA pH 8.0 |
| | 0.25% bromophenol blue |
| | 25% xylene cyanol FF |
| SSC (20×) 3 M NaCl | 300 mM sodium citrate |
| TAE (50×) | 2 M tris acetate |
| | 50 mM EDTA |
| TBE (10×) | 1 M tris-borate |
| | 10 mM EDTA |
| TE | 10 mM Tris HCl, pH 7.4 |
| | 1 mM EDTA |
| TES | 10 mM Tris HCl, pH 7.5 |
| | 1 mM EDTA |
| | 0.5% SDS |

1.3 Electronic Data Processing Programs

| | |
|---|---|
| ImaGene | |
| ImageQuant | |
| Excel | |
| 1.4 Enzymes | |
| Restriction enzymes: | Boehringer Mannheim EcoRV |
| | Gibco Life Technologies Bg1II |
| | New England Biolabs NsiI |
| Modification enzymes: | Gibco Life Technologies |
| | Taq polymerase |
| | SuperscriptII |
| | New England Biolabs |
| | (rev. Transcriptase |
| | T4-polynucleotide kinase |
| 1.5 Fluorescent Dyes | |
| Amersham: | Cy 3-dCTP, Cy 5-dCTP |
| 1.6 Devices | |
| Array scanner: | Genetic Micro Systems 418 |
| | Cambridge, MA, USA |
| Array spotter: | Genetic Micro Systems 417 |
| | Cambridge, MA, USA |
| Incubators: | Heraeus FunktionLine B12 |
| | Memmert BF40 |
| Electrophoresis: | Amersham Pharmacia Biotech EPS |
| | 301 Netzteil |
| | Easy-Cast Electrophoresis System #B1A |
| | Hoefer Max Submarine Unit HE 99X |
| Gel Documentation: | Fröbel Labortechnik |
| Heating blocks: | Laboratory Devices Digiblock |
| Hybridization oven: | GFL 7601 |
| Orbital shaker: | Belly Dancer, Stovall Life Science |
| pH meter: | Griesinger Electronics Mess- und Regeltechnik |
| Phospho-imager | Molecular Dynamics Storm 860 |
| | Molecular Dynamics Phosphor Screen and |
| | Exposure Cassette |
| Photometer | Beckman DU-62 |
| Shaking incubator: | HAT Infors GmbH |
| Thermocycler: | Hybaid Touchdown |
| UV crosslinker: | Stratagene UV-Stratalinker 1800 |
| Vortexer: | Gemmy Industries VM-300 |
| Balances: | Sartorius BP 410 |
| | Sartorius BP 121S |
| Water baths: | Medingen W 12 |
| Centrifuges: | Heraeus Megafuge 1.0R, refrigerated |
| | centrifuge |
| | Heraeus Megafuge 1.0 |
| | Heraeus Biofuge pico |
| | Heraeus Bifuge fresco, refrigerated table |
| | centrifuge |
| 1.7 Isotopes | |
| Amersham: | [γ-³²P]-ATP 18.5 MBq |
| | [α-³²P]-dCTP 9.25 MBq |
| 1.8 Media | |
| YPD medium: | Complete medium for yeasts |
| | Yeast extract (Difco) 10 g/l |
| | Bacto-Peptone (Difco) 20 g/l |
| | Glucose solution (40%) 50 ml/l |
| SC medium: | Synthetic medium for yeasts |
| | Difco YNB 1.5 g/l |
| | Ammonium sulfate 5.0 g/l |
| | Glucose solution (40%) 50 ml/l |
| | Amino acids, Nucleotides |
| SC-ura medium: | Selection medium for yeasts |
| | like SC medium, without uracil |
| Complete media: | RPMI-1640 (Gibco Life Technologies) |
| | α-MEM (Gibco Life Technologies) |
| SC-ura agar plates: | Addition of equal volume of 4% agar |
| | to the SC-ura medium |

1.9 Plasmids

pGEM-T [Promega]

p275 [26]

1.10 Primers

For the production of the DNA chip, gene sequences of cell walls in Saccharomyces cerevisiae were run with the Candida database and homologous sequences in Candida albicans were thus found. These sequences, and also sequences of known cell wall genes in Candida albicans, formed the basis for primer selection. The primers used are given in Appendix 6.3 on pages 63-65.

1.11 Consumable Materials

| | |
|---|---|
| Amersham: | Hybond-N nylon membrane |
| | ProbeQuant G-50 Micro Columns |
| | ProbeQuant G-25 Micro Columns |
| Eppendorf: | 1.5 ml reaction vessels (RNAse free) |
| Greiner: | PP-tubes 15 or 50 ml, sterile |
| Qiagen: | QIAquick Gel Extraction Kit |
| | QIAquick PCR Purification Kit |
| Roth: | Whatman paper, 190 g/m², |
| | thickness 0.37 mm |
| Sigma Diagnostics: | Glass microscope slides |
| Stratagene: | Prime-it II Random Primer Labeling Kit |
| Gibco BRL: | Superscript II RT Kit |
| Amicon: | Microcon-30 Filter |
| Millipore: | Millipore Filter, 0.45 μm |

2. Methods

2.1 RNA Isolation from Candida albicans

The method of acid phenol extraction at 65° C. was used here for RNA isolation. The cultured cells were centrifuged off at 1500×g and 4° C. for 15 minutes, media residues were washed out with ice-cold ddH₂O, and the cells were resuspended in a 1:1 mixture of TES and phenol. The amount of this mixture corresponded to the volume of the cell pellet. After incubation for 60 minutes at 65° C., with frequent mixing to increase the RNA yield, two phases were formed by further centrifugation at 1500, g and 4° C. for 15 minutes. The aqueous upper phase in which the RNA had collected was taken off from the lower phenol phase, pipetted into a like amount of prepared phenol, and once again incubated for 15 minutes at 65° C. The subsequent phase separation took place as already described. In order to remove residual amounts of phenol from the aqueous phase, it was pipetted into a like volume of chloroform and vigorously mixed. Further centrifugation at 1500×g and 4° C. for 15 minutes gave two clearly separated phases, the upper aqueous phase again being added to a prepared mixture of 100% ice-cold ethanol and 0.1 volume of 3M sodium acetate, pH 5.3. Under these conditions, the RNA precipitated after 2-24 hours and could be centrifuged off at 1500×g and 4° C. for 15 minutes. While residual salts dissolved out by washing once in ice-cold 70% ethanol, the RNA remained in its undissolved pellet form. The supernatant was rejected and residual amounts of ethanol were removed by air-drying. The RNA was finally resuspended in an amount of DEPC-H₂O to give a final concentration of 5-10 μg/μl.

2.2. LiCl Precipitation

Isolated RNA was reprecipitated overnight for labeling with fluorescent dyestuff in a 1:1 ratio with 4M LiCl. After centrifuging off the precipitated RNA at 1500×g and 4° C. for 45 minutes, three washings took place with 70% ethanol in order to separate off residual amounts of salt which could interfere with reverse transcription. Air drying then removed the residual traces of ethanol. Thereafter the RNA was again resuspended in DEPC-H₂O to a final concentration of 5-10 μg/μl.

### 2.3 Determination of RNA Concentration

RNA concentrations were determined by photometric extinction measurement at 260 nm and subsequent calculation. The calculation took place by means of the dilution used and a conversion factor, in which an extinction of 1.0 was based on a RNA concentration of 40 μg/ml. The quotient of E260/E280 was used for purity determination. The quotient had to be between 1.8 and 2.0 for sufficiently pure RNA.

2.4 Gel Electrophoresis

(a) RNA Gels

Agarose gels of the following composition were used for RNA gel electrophoresis:

1% agarose

72% ddH₂O

10% MOPS (10×)

18% formaldehyde (12, 3 M)

1 μl ethidium bromide (10 μg/μl).

The sample formulation was prepared as follows:

| | |
|---|---|
| RNA (15 μg) | 5.0 μl |
| MOPS (10×) | 1.5 μl |
| Formaldehyde (12.3 M) | 2.5 μl |
| Formamide | 7.5 μl |
| RNA loading buffer | 2.5 μl |
| DEPC-H₂O | 0.8 μl |

Electrophoresis was performed under the following conditions:

| | |
|---|---|
| Gel running: | at 10 V, 400 mA overnight, 14-16 hours |
| | or at 70 V, 400 mA for 4 hours |
| Loading buffer: | 1 × MOPS |
| (b) DNA Gels | |

DNA gel electrophoresis was likewise performed with 1% agarose gels, set in 1×TBE buffer. The ethidium bromide was added to the gels in a proportion of 1:15,000 of a 10 μg/μl original solution. The sample formulation took place in a proportion of 1:1 with the sample buffer. The electrophoresis conditions were as follows:

| | |
|---|---|
| Gel running: | at 100-120 V, 400 mA for 45 minutes to 1 hour |
| Running buffer: | 1 × TBE buffer |
| Sample buffer: | DNA loading buffer (2×) |

(c) DNA Gel for Testing the Probes for DNA Chip

For testing the PCR products in chip production, a 1.2% agarose gel was used, formulation taking place in 1×TAE buffer. The ethidium bromide was added to the gel at point (b) DNA gels, and the sample buffer was added to the sample in a proportion of 1:1. The conditions were chosen as follows:

| | |
|---|---|
| Gel running: | at 150 V, 400 mA for 1.5 hours |
| Running buffer: | 1 × TAE buffer |
| Sample buffer: | DNA loading buffer (2×) |

2.5 Restriction Digestion

Restriction digestion with NsiI took place in a volume of 30 μl with 3 μl P275-DNA (about 1 μg) and 4 U enzyme at 37° C. Incubation took place overnight.

2.6 PCR

The DNA fragments used for the trials were amplified by means of the polymerase chain reaction (PCR). The formulations contained respectively 10 ng DNA, 1×PCR buffer (Gibco), 1.5 mM MgCl₂, 0.2 mM of each nucleotide, 20 pmol of each primer, and 2 U Taq polymerase in a volume of 50 μl.

The program course of the cycler differed in annealing temperature according to the primer pair used. For PCR amplification, as used for chip production on the 96-well plate scale, the following was used:

Probes:

| | | |
|---|---|---|
| Denaturing at 95° C. | 1 min | |
| Annealing primer at 52° C. | 1 min | 30 cycles |
| Elongation at 72° C. | 1 min | 30 cycles |
| further denaturation at 95° C. | 1 min | 30 cycles |
| Cooling to 4° C. | until removal from device | 30 cycles |

Exceptions were ASL1, PHR1, PHR2, p18b. For the amplification of these probes, the annealing temperatures had to be changed as follows:

| | |
|---|---|
| ASL1 | 1 min at 55° C. |
| PHR1, PHR2, p18b | 1 min at 50° C. |

For the amplification of ACT1, 2436 (HWP1) and 2343 (ALS 1, 3, 8), which were used as template DNA in the production of the radioactive probes, a PCR was performed without radioactive labeling (see 2.2.10). The conditions were as follows:

| | | |
|---|---|---|
| Denaturing at 95° C. | 1 min | |
| Annealing primer at 50° C. | 1 min | 30 cycles |
| Elongation at 72° C. | 1 min | 30 cycles |
| further denaturation at 95° C. | 1 min | 30 cycles |
| Cooling to 4° C. | until removal from device | 30 cycles |

2.7 Purification of DNA Fragments

The purification of the DNA fragments from PCR and restriction digestion took place in general according to the protocol of the Qiagen QIAquick PCR Purification Kit. The obtained DNA was eluted in respectively 30-50 μl of microbiologically pure water.

2.8 Production of Radioactive Probes

(a) Labeling with [α-³²P]-dCTP

The production of the probes radioactively labeled with [α-³²P]-dCTP and used for the Northern blot took place according to the protocol of the Stratagene Prime-It II Random Primer Labeling Kit. [α-³²P]-dCTP was thereby built in as a radioactive nucleotide in the synthesis chain. A subsequent separation of radioactive nucleotide which had not been built in was performed according to the protocol of the Amersham ProbeQuant G-50 Micro Columns.

(b) Labeling with [γ-³²P]-ATP

The production of the oligo(dT) probes radioactively labeled with [γ-³²P]-ATP and used for mRNA detection took place by the conversion of oligo(dT) primers with T4-polynucleotide kinase according to the specification of New England Biolabs. Here a transfer of the radioactive y ³²P from ATP to the oligo(dT) primer took place by means of T4 polynucleotide kinase. The formulation contained the following:

| | |
|---|---|
| 0.5 μl | oligo(dT) primer (2 μg/μl) |
| 12.5 μl | [γ-³²P]-ATP (10 mCi/ml) |
| 5.0 μl | polynucleotide kinase buffer (10×) |
| 2.0 μl | T4-polynucleotide kinase (10,000 U/ml) |
| 30.0 μl | DEPC-H₂O |

Incubation times were 30 minutes at 37° C. The separation of unconverted radioactive nucleotide took place by means of G-25 Micro Columns according to the protocol of Amersham ProbeQuant.

2.9 Northern Blot Analyses

Northern blot analyses were performed as described by Srikantha et al., (Mol. Gen. Genet. (1995) 246, 342-352). 10 mg total RNA was separated on a 1.1% agarose gel which contained 2.2 M formaldehyde and 0.5×MOPS, pH 7.0. After staining with ethidium bromide, the gels were blotted on Biodyne B-Nylon membrane (PALL FILTRON, Germany).

Hybridization was performed at 62-65° C. with radioactively labeled DNA fragments; the membranes were then treated and autoradiography was performed as usual (Church and Gilbert, Proc. Natl. Acad. Sci. USA, 81 (1984), 1991-1995). The Northern blots were quantified by means of a Fuji BAS 3000 Phosphor IMAGER Device using the associated software. The Southern blot analyses were performed as described by Schroppel et al., (J. Clin. Microbiol., 32 (1994), 2646-2654). Genomic DNA cleaved with restriction enzymes was separated on 0.7% agarose gels and blotted on Biodyne B-Nylon membranes. The hybridization and stringent washing steps were performed as described (Church and Gilbert (1984), loc. cit.) followed by autoradiography.

For the detection of CaTEC1, a NsiI fragment of p275 was labeled with random primers. For the detection of SAP4-6, an equimolar mixture of p206, p207 and p208 was labeled which was produced by subcloning of BgIII fragments of the SAP4-, SAP5- and SAP6-ORFs (Monod et al., Mol. Microbiol., 13 (1994), 357-368) in the BamHI site of pGEM3Z (length of the insert: 755, 677 and 660 bp). Plasmids for the detection of ACT1- and EFG1-mRNA were kindly made available by Prof. J. Ernst, Heinrich-Heine Universität, Düsseldorf.

Northern blots for the detection of genes regulated by CaTEC1 were performed as follows.

(a) Transfer and Immobilization of the RNA

For the Northern blots, 15 mg of the respective RNA isolated from strains was mixed with RNA loading dye in a proportion of 1:3 and was separated overnight on a 1% RNA gel at 10 V in 1×MOPS. For monitoring, the gel was photographed under UV light and then placed with its top side downward on a blot block, cut to fit, of Whatman paper to which 20×SSC had been previously added in a blot tray as transfer buffer. In order to ensure a capillary action through the gel and not through any overhanging paper, the edge of the gel was covered with Parafilm. A nylon membrane cut exactly to the gel size and previously wetted in 2×SSC was placed, free from air bubbles, on the gel and covered with 3 layers of Whatman paper. A stack of paper towels 5 cm thick was laid thereover and weighted with a flat dish and a weight. This provided for optimum capillary action. The RNA present in the gel could thus be transferred to the nylon membrane overnight, and after air drying could be fixed by means of “auto cross link” in the UV crosslinker. Subsequent washing of the volt in 2×SSC should reduce the high salt concentration on the membrane. The membrane was blocked in the hybridizing oven at 65° C. with the blot in foil after again air drying. The hybridization proper then took place with the radioactively labeled probe, previously placed in 10 ml of Church buffer. In order to ensure optimum hybridization, this was performed overnight at 65° C.

(c) Washing Steps

After washing three times in 1×SSC with 0.1% SDS for 15 minutes each at 65° C., the membrane in the wet state was sealed in foil and was placed on a phosphor screen (Molecular Dynamics).

(d) Detection and Documentation

The exposure period varied, but took place at least overnight. It was then sealed and stored at −70° C. until further treatment in the isotope laboratory.

(b) Pre-Hybridization and Hybridization

In order to saturate the free binding places on the blot membrane and thus to guarantee a specific hybridization of the probe, pre-hybridization with Church buffer was necessary. After 1-2 hours of incubation, the screen was scanned on the Phosphoimager. Quantifying by means of the ImageQuant software gave the raw data, which were a prerequisite for further evaluation.

2.10 Invasion Tests and Pseudohyphal Growth in S. cerevisiae

Invasive and pseudohyphal growth test were performed as described (Gimeno and Fink, Mol. Cell. Biol., 14 (1994), 2100-2112; Robertson and Fink, Proc. Natl. Acad. Sci. USA, 95 (1998), 13783-13787). The invasive growth was tested in that cells grown on selective medium were transferred to YPD plates, incubated for 24 or 48 hours at 30° C., and then washed. Pseudohyphal growth of diploid strains was tested in that the strains as individual colonies were plated out on SLAD agar, and these plates were incubated for 2 days at 30° C.

2.11 Production of Extracts and Enzyme Tests

The cells for the β-galactosidase test of FLO11::lacZ or FG::TyA-lacZ were allowed to grow in SC liquid medium and were quantified according to Mösch et al. (Proc. Natl. Acad. Sci. USA, 93 (1996), 5352-5356). Cells for the quantification of FLO11::lacZ or FG::TyA-lacZ expression in the exponential growth phase were inoculated as confluent 20-hour cultures 1:20 into fresh medium and allowed to grow for 4-6 hours. The cells for the quantification of FLO11::lacZ and FG::TyA-lacZ expression after the post-diauxic shift were allowed to grow for 48 hours.

2.12 Interaction of C. albicans and Macrophages (MΦ)

The interaction between C. albicans and MΦ was investigated in that MΦ obtained from peritoneal exudates were used, and were produced substantially as described in Bogdan et al. (Eur. J. Immunol., 20 (1990), 1131-1135; Gessner et al., Infect Immun., 61 (1993), 4008-4012). MΦ were set out at a density of 8×10⁵ cells on plates having eight depressions (Nunc, Germany) and formed a monolayer of adherent cells. C. albicans was added at an infection multiplicity (MΦ, multiplicity of infection) of 1:16 (C. albicans: MΦ ratio), and the plates were incubated at 37° C. with 5% CO² for 8 or 24 hours. The plates were fixed (Histo Choice, Amresco) and stained with periodic acid—Schiff reagent (Sigma). Microscopic investigations were then performed with a Zeiss Axiophot microscope (Zeiss, Göttingen).

2.13 Virulence Studies

Eight- to ten-week-old female BALB/c mice (Charles Rivers Breeding Laboratories, Sulzfeld, Germany, 5-8 per group) were used in the in vivo studies. The mucosal colonization of the vaginal canal was initiated by inoculating BALB/c mice intravaginally with 5×10⁴ blastoconidia of the stationary phase in 20 μl PBS (Fidel, Jr. et al., Infect Immun (1993) 61, 1990-1995). 72 hours before inoculation, the mice were injected subcutaneously with 0.02 mg per mouse of estradiol valerate (Sigma) in 0.1 ml of sesame oil. The estrogen treatments were repeated at weekly intervals. At days 10 and 24 after the estrogen treatment, corresponding to 7 and 21 days after inoculation, the animals were killed and the vagina of each mouse was flushed with 100 μl of PBS. The fungal presence in the vagina was determined by a vaginal flushing culture as described by Fidel et al., ((1993), loc. cit), and a portion of the recovered liquid was used for microscopic investigation. After incubation at room temperature for 48-72 hours, the colony forming units (CFU) were determined. For systemic infections, groups of 10 mice were injected with 5×10⁵ living cells by intravenous injection and the survival rate was determined (Csank et al., (1997 and 1998) loc. cit.; Timpel et al., J. Bacteriol., 182 (2000), 3063-3071). The survivability of the infected population was determined by plating out and counting the CFU. Survival curves were prepared by means of the Kaplan-Meier method using the PRISM program (GraphPAD Software, San Diego, USA) and were compared with the log rank test.

For the microscopic investigation of the cellular morphology of C. albicans during the infection, fungal cells were isolated from the vaginal canal (day 21) or from the kidneys (day 12). The samples were dissolved using a 20% KOH solution. Samples were centrifuged for 10 minutes at 1500×g, and the sediment was transferred to slides in the presence of fluorescent brighteners (Calcofluor White, Sigma, Germany) for epifluorescence microscopy (Zeiss Axiphot, Zeiss, Germany).

2.14 Chip Production

Chip production was performed according to the protocols of Stanford University [31].

(a) Slide Preparation

Glass slides (hereinafter referred to as slides) were cleaned on an orbital shaker for 2 hours with a solution consisting of 95% ethanol and NaOH, as described in protocol [32]. Subsequent washing five times in respectively fresh ddH₂O was to completely remove residual NaOH. Further coating with a poly-L-lysine solution, which was freshly prepared shortly before, took place for an hour, again on the orbital shaker. After washing again in ddH₂O, the slides were dried by centrifuging at 150×g for 5 minutes and then incubation at 45° C.

(b) Printing of the Probes

Selection criteria for the probes which were to be printed on this chip were homologies in Candida albicans to cell wall genes in Saccharomyces cerevisiae, and also known cell wall genes in Candida albicans. These probes were amplified by PCR on a 96-well plate scale and then purified. A gel run of a 1.2% agarose gel at 150 V for 1.5 hours gave a conclusion as to the quality of the probes. Printing onto the coated slides took place on the spotter.

(c) Post-Processing of the Slides

After printing of the slides, the printed region had to be marked before each working step with a glass marker in order to be able to later position the cover glass precisely for the hybridization. It was rehydrated on the slide so that a uniform distribution of the DNA in each individual spot could be ensured. Here the probes were allowed to swell briefly by means of 1×SSC in order to dry them again at 80° C. for 3 seconds. Subsequent fixing of the DNA on the slides took place by the formation of covalent bonds by means of UV irradiation in the UV Crosslinker. In order to block the still remaining free lysing groups and thus to avoid unspecific binding of the samples to be investigated, the slides were treated for 20 minutes with a blocking solution, which contained, as described in protocol [33], succinic acid, sodium borate, and N-methylpyrrolidone. The DNA on the slides was thereafter denatured, in that the slides were left in a 95° C. water bath for 2 minutes. Drying of the slides was performed by washing in 95% ethanol and then centrifuging at 150×g for 5 minutes.

1. Nucleic acid molecule, comprising a nucleic acid molecule which is suitable for the cloning of a gene coding for a transcription factor and/or codes for a protein with the biological activity of a transcription factor, chosen from the group consisting of:
(a) a nucleic acid molecule defined in SEQ ID No.1, 2, 3 or 7, a complementary strand or portion thereof,
(b) a nucleic acid molecule coding for the amino acid sequence defined in SEQ ID No. 4, a complementary strand or a portion thereof,
(c) nucleic acid molecule which represents a derivative, an allelic variation and/or a homolog of the nucleic acid molecule of (b),
(d) a nucleic acid molecule, obtainable from a gene bank of Candida albicans by using the primers shown in SEQ ID No. 5 and SEQ ID No. 6 by means of the PCR method, and
(e) a nucleic acid molecule which hybridizes with one of the nucleic acid molecules named under (a), (b), (c) or (d).

2. Nucleic acid molecule according to claim 1, which is a regulating element and is defined in SEQ ID No. 3 or 7.

3. Nucleic acid molecule according to claim 1, which contains a nucleotide sequence coding for a protein and defined in SEQ ID No. 4.

4. Nucleic acid molecule according to one of the foregoing claims, which is a DNA or RNA molecule.

5. Nucleic acid molecule according to one of the foregoing claims, which is suitable for the cloning and/or expression of a fungal transcription factor.

6. Nucleic acid molecule according to claim 5, wherein the fungus is Candida albicans.

7. Vector, including a nucleic acid molecule according to one of claims 1-6.

8. Vector according to claim 7, wherein the nucleic acid molecule is under the operative control of at least one regulating element which ensures the expression of a translatable RNA in pro- or eukaryotic cells.

9. Vector according to claim 8, wherein the regulating element is a promoter, enhancer, silencer or 3′-transcription terminator.

10. Vector according to one of claims 7-9, wherein the regulating element is a signal sequence for localization of the protein coded for by the nucleic acid molecule within given cell organelles or compartments or in the extracellular space.

11. Vector according to one of claims 7-10, wherein the vector is a plasmid, cosmid, bacteriophages or virus.

12. Plasmid according to claim 11, deposited in Escherichia coli at the DSMZ in Braunschweig, Germany, under the number DSM 13716.

13. Host cell, containing a vector according to one of claims 7-12.

14. Host cell according to claim 13, wherein the host cell is a pro- or eukaryotic host cell.

15. Host cell according to claim 14, wherein the host cell is a bacterial cell, yeast cell, insect cell, or mammalian cell.

16. Escherichia coli cell according to claim 15, containing a plasmid according to claim 12, deposited at the DSMZ in Braunschweig, Germany, under the number DSM 13716.

17. Candida albicans cell, deposited at the DSMZ in Braunschweig, Germany, under the number DSM 13722.

18. Method for the production of a transcription factor, wherein a host cell according to one of claims 13-17 is cultured in a suitable culture medium under conditions which permit the expression of the transcription factor, and this is recovered.

19. Protein with the biological activities of CaTEC1 and an amino acid sequence shown in SEQ ID No. 4.

20. Protein produced according to the method according to claim 18.

21. Antisense RNA sequence which is complementary to a mRNA which is transcribed by a nucleic acid according to one of claims 1-6, and can selectively bind to the mRNA or a portion thereof, wherein the antisense RNA sequence can inhibit the synthesis of the protein coded for by the nucleic acid molecules.

22. Ribozyme, which can bind selectively to the mRNA which is transcribed by a nucleic acid molecule according to one of claims 1-6 or a portion thereof and can cleave this, wherein the synthesis of the protein coded for by the nucleic acid molecule is inhibited.

23. Inhibitor which can suppress the activity of a protein according to claim 19 or 20.

24. Antibody which specifically recognizes and binds a protein according to claim 19 or 20.

25. Antibody according to claim 24, which is a monoclonal, polyclonal, and/or modified antibody.

26. Antibody which is directed against an antibody according to claim 25.

27. Method for the diagnosis of a Candida infection, comprising bringing into contact a sample which is suspected of containing CaTEC1 protein and/or a nucleic acid coding for CaTEC1, with a substance which reacts with CaTEC1 and/or with a nucleic acid which codes for CaTEC1, and the detection of CaTEC1 and/or of the nucleic acid coding for CaTEC1.

28. Method according to claim 27, wherein the substance is a CaTEC1-specific nucleic acid probe.

29. Method according to claim 28, wherein the CaTEC1-specific nucleic acid probe comprises the nucleic acid sequences 5′-TTT AGG ATC CAA TGA TGT CGC AAG CTA CTC C-3′ and 5′-TTT AGG ATC CAC TAA AAC TCA CTA GTA AAT CCT TCT G-3′.

30. Method according to claim 27, wherein the substance is an antibody directed against CaTEC1.

31. Method according to one of claims 27-30, wherein the substance is detectably labeled.

32. Method according to claim 31, wherein the labeling is selected from the group consisting of a radioisotope, a bioluminescent compound, a chemiluminescent compound, a fluorescent compound, a metallic chelate, or an enzyme.

33. Method for the treatment of a Candida infection, comprising the administration of a therapeutically effective amount of a substance which diminishes or inhibits the expression of CaTEC1 or the activity of CaTEC1, to a mammal.

34. Method according to claim 33, wherein the Candida infection is a mucosal or a systemic infection.

35. Method according to claim 33 or 34, wherein the substance is a nucleotide sequence which includes an antisense RNA.

36. Method according to claim 33 or 34, wherein the substance is a nucleotide sequence which includes a ribozyme.

37. Method according to claim 19 or 20, wherein the substance is a inhibitor of CaTEC1.

38. Method according to claim 37, wherein the inhibitor is an antibody or a fragment thereof directed against CaTEC1.

39. Diagnostic composition, containing a nucleic acid molecule according to one of claims 1-6, a vector according to one of claims 7-12, a protein according to one of claims 19 or 20, an antisense RNA according to claim 21, a ribozyme according to claim 22, an inhibitor according to claim 23, and/or an antibody according to one of claims 24-26.

40. Pharmaceutical composition, containing a nucleic acid molecule according to one of claims 1-6, a vector according to one of claims 7-12, a host cell according to one of claims 13-17, a protein according to one of claims 19 or 20, an antisense RNA according to claim 21, a ribozyme according to claim 22, an inhibitor according to claim 23, and/or an antibody according to one of claims 24-26, in necessary together with a pharmaceutically acceptable carrier.

41. Diagnostic kit, suitable for the detection of a Candida infection, containing a nucleic acid molecule according to one of claims 1-6, a vector according to one of claims 7-12, a host cell according to one of claims 13-17, a protein according to one of claims 19 or 20, an antisense RNA according to claim 21, a ribozyme according to claim 22, an inhibitor according to claim 23, and/or an antibody according to one of claims 24-26.

42. Diagnostic kit, containing a nucleic acid molecule according to one of claims 1-6, which specifically hybridizes with CaTEC1 gene sequences and/or a CaTEC1 specific antibody or a CaTEC1 binding fragment thereof.

43. Use of a nucleic acid molecule according to one of claims 1-6, a vector according to one of claims 7-12, a host cell according to one of claims 13-17, a protein according to one of claims 19 or 20, an antisense RNA according to claim 21, a ribozyme according to claim 22, an inhibitor according to claim 23, and/or an antibody according to one of claims 24-26, for the production of a medicament for the treatment of diseases brought about by species of the genus Candida.

44. Method for finding and identifying therapeutically effective substances against diseases brought about by species of the genus Candida, wherein a substance to be tested is brought into contact in a suitable medium with at least one agent chosen from the group consisting of a nucleic acid molecule according to one of claims 1-6, a vector according to one of claims 7-12, a host cell according to one of claims 13-17, a protein according to one of claims 19 or 20, an antisense RNA according to claim 21, a ribozyme according to claim 22, an inhibitor according to claim 23, and/or an antibody according to one of claims 24-26, and an interaction between the substance to be tested and one of the said agents is detected.

45. Method according to claim 44, wherein the binding of the substance to be tested to a double-stranded nucleic acid molecule according to one of claims 1-6 is detected.

46. Method according to claim 44, wherein the substance to be tested is an oligonucleotide or a derivative thereof.

47. Method according to claim 46, wherein the binding depends on the formation of a DNA triple helix.

48. Method according to claim 45, wherein the substance to be tested is a protein nucleic acid (PNA) molecule.
